(19) United States
(12) Patent Application Publication
(10) Pub. No.: US 2025/0367373 A1
(43) Pub. Date: Dec. 4, 2025

(54) PLUNGER RODE RELEASABLY ATTACHABLE TO PLUNGER HEAD IN A PUMP DEVICE

(71) Applicant: NeuroDerm, Ltd., Rehovot (IL)

(72) Inventor: Lior Bar Chen, Ganei Tal (IL)

(21) Appl. No.: 18/873,842

(22) PCT Filed: Jun. 5, 2023
(86) PCT No.: PCT/IL2023/050577

§ 371 Date: Dec. 11, 2024

Related U.S. Application Data
(60) Provisional application No. 63/352,362, filed on Jun. 15, 2022.

(51) Int. Cl. A61M 5/145; A61M 5/315

(52) U.S. Cl. CPC A61M 5/14546; A61M 5/1456; A61M 5/31515; A61M 2005/14573

(57) ABSTRACT

A disposable reservoir includes a plunger head that comprises a PEM and bidirectionally moveable in unison with the PEM between a proximal end of the disposable reservoir and a distal end of the disposable reservoir. The PEM is configured to engage a plunger rod of a reusable part of a drug delivery device. The disposable reservoir also includes a PRM that is configured to release the plunger rod from the PEM upon retraction of the PEM to a stow position in the PRM. The plunger rod may be locked by the PEM and released from the PEM by using a snap-fit feature. Alternatively, the plunger rod may be locked by the PEM by using a snap-fit feature and released from the PEM by using a bayonet connection means. Alternatively, the plunger rod may be both locked by the PEM and released from the PEM by using a bayonet connection means.

### FIELD OF THE INVENTION

The present invention generally relates to systems and methods for coupling a disposable part (“DP”) (e.g., liquid drug reservoir) of a drug delivery device (e.g., pump device) to a reusable part (“RP”) of the pump device. More specifically, the present invention relates to a plunger rod and plunger head designs that enable to releasably engage the plunger rod with the plunger head.

### BACKGROUND

Some liquid drug delivery systems are two-part systems including a reusable part, which typically includes, among other things, an electric motor and a gear system that is driven by the electric motor, and a disposable part that typically includes a liquid drug reservoir and a gear-driven plunger, or a standalone plunger, means to expel drug from the reservoir. In conventional drug delivery devices, the plunger means includes a plunger rod (sometimes referred to as “leadscrew” or spindle) and a plunger head, and the plunger rod is an inseparable part of the plunger head. In addition, such a plunger means is usually inseparable from the reservoir itself, so, using such a plunger means has drawbacks. For example, the disposable reservoir is disposed of after use together with the plunger rod and plunger nut (a means whose rotational movement is converted into linear movement of the plunger rod), or other/additional driving elements, which is wasteful and not economic. In addition, since the plunger rod (the leadscrew) conventionally resides in the disposable reservoir, coupling between the disposable reservoir and the reusable part oftentimes results in degraded accuracy of the medicament delivered due to some uncertainty of the precise location, or position, of the leadscrew in the reservoir. In addition, the assembly process of a disposable reservoir that includes a plunger rod is relatively complicated. Furthermore, inseparably affixing a plunger rod to disposable reservoirs prevents storing or distributing such disposable reservoirs as ‘pre-filled’ disposables. Therefore, conventional disposable reservoirs are typically filled up from a vial only a short time prior to them being used.

### SUMMARY OF THE INVENTION

A pump device for delivering medicament includes a disposable reservoir and a reusable part that is detachably couplable to the disposable reservoir. The disposable reservoir includes, among other things, a plunger head that is bidirectionally moveable in the disposable reservoir between a proximal end of the disposable reservoir and a distal end of the disposable reservoir. The reusable part includes, among other things, a plunger rod. The plunger head and the plunger rod are designed such that when the disposable reservoir and the reusable part are coupled to one another the plunger rod is releasably lockable in the plunger head as the plunger rod moves linearly, optionally bi-directionally (back and forth), in the disposable reservoir in unison with the plunger head. (“Distal end” of the disposable reservoir is the end of the reservoir that includes an injection opening through which liquid medicament can move into the reservoir, e.g., from a vial, in order to fill up the reservoir, and outside the disposable reservoir when the medicament is expelled out of the reservoir, to a patient. “Proximal end” of the disposable reservoir is the end of the reservoir opposite the distal end of the disposable reservoir. So, to fill the disposable reservoir up with medicament, a plunger head is to be longitudinally (axially) moved inside the disposable reservoir from the reservoir's distal end to the reservoir's proximal end, and in the opposite direction to deliver the medicament from the reservoir to a patient.

The plunger head includes a concentric plunger engagement member (PEM) that is bidirectionally moveable in unison (en masse) with the plunger head between the proximal end of the disposable reservoir and the distal end of the disposable reservoir, and the disposable reservoir further comprises a bored plunger release member (PRM) that is concentrically and fixedly mounted to the proximal end of the disposable reservoir. The plunger rod includes a distal end and is bidirectionally moveable through the bored PRM. When the disposable reservoir and the reusable part are coupled to one another the distal end of the plunger rod is lockable in the PEM and releasable from the PEM by the PRM. The PRM, by functioning both as an ejection means and as a stowing means, ejects the plunger rod from the PEM when the plunger rod pulls the PEM into a stow position in the PRM. The plunger rod may be lockable in and releasable from the PEM by using a snap-fit connection means, or a bayonet connection means, or a combination of snap-fit connection means and bayonet connection means. The snap-fit connection means may include an annular snap-fit feature, or a cantilever snap-fit connection feature, or a combination of annular snap-fit feature and cantilever snap-fit connection feature.

### Configuration #1: Locking and Releasing by ‘Snap-Fit’

In some embodiments the plunger rod is lockable in the PEM and releasable from the PEM by using a snap-fit connection means. The snap-fit connection means may include the distal end of the plunger rod, the PEM and the PRM. The snap-fit connection is a non-reversible snap-fit connection, meaning that once the plunger rod is locked in/by the PEM by snap-fitting it to the PEM, the plunger rod can be released from the PEM only by using a ‘third party’ (i.e., the PRM).

The distal end of the plunger rod includes an entrance tip (plunger interface) and an annular lock groove that is circumferentially formed in the distal end between the entrance tip and the rest of the plunger rod. The PEM includes a ring-shaped base and elongated flexible arms that circularly extend from the ring-shaped base and bend inwardly towards a central axis of the PEM to, thus, form a flexible cap-like structure with a central opening. The flexible cap-like structure is designed to be snap-fit table into the annular lock groove of the plunger rod.

The PRM includes an external (outer) cylindrical body and a concentric inner (internal) hollow cylindrical body through which the plunger rod can be moved bidirectionally, for example by an electric motor that is included in the reusable part. The external cylindrical body and the concentric inner hollow cylindrical body define, therebetween, an open annular channel to accommodate the PEM, and the inner hollow cylindrical body of the PRM is configured to deflect the elongated flexible arms of the PEM radially outward to release (to ‘snap-out’) the plunger rod from the PEM when the PEM is retracted by the plunger rod into a rest (stow) position in the open annular channel in the PRM. The entrance tip of the plunger rod may be selected from the group consisting of hemispherical tip, conical tip, and truncated trapezoid.

Each of the elongated flexible arms of the PEM may include a proximal section that extends from the ring-shaped base at an acute angle α relative to a plane of the ring-shaped base (where 0°<α<90°, for example α=80°). Each elongated flexible arm of the PEM may additionally include a distal section that contiguously (and seamlessly) extends from the corresponding proximal section at an acute angle β relative to the ring-shaped base (where 0°<β<α, for example β=60°). The distal sections of the elongated flexible arms are configured to snap-fit (lock) into the annular lock groove of the plunger rod when the entrance tip of the plunger rod is linearly pushed against the elongated flexible arms of the PEM.

The annular lock groove of the plunger rod includes an annular pulling ledge for pulling the PEM in unison with the plunger rod in a backward (refilling) direction by pulling the distal sections of the elongated flexible arms when the plunger rod is retracted in the disposable reservoir. The annular lock groove may also include a conical surface to enable movement of the plunger rod through the opening in the flexible cap-like structure in a forward (emptying) direction until the entrance tip of the plunger rod contacts (abuts) an inner push surface of the plunger head. As the entrance tip of the plunger rod is moved forward through the opening in the flexible cap-like structure, the elongated flexible arms are deflected radially outward by the entrance tip of the plunger rod and, as the entrance tip is moved passed the opening in the flexible cap-like structure, the flexible cap-like structure is snap-fitted into the annular lock groove of the plunger rod in a way that locks the plunger rod in the plunger engagement member (PEM). The entrance tip of the plunger rod may include a curved entrance side to facilitate deflection of the elongated flexible arms radially outward.

The number of the elongated flexible arms may be 2n (where n=1, 2, 3, . . . ), and the elongated flexible arms may be equally sized and shaped, and may be angularly equidistant from one another; namely, the elongated flexible arms may be angularly and equidistantly distributed around a longitudinal axis of the PEM. The number of elongated flexible arms may be selected, for example, from the group consisting of four (4) elongated flexible arms, six (6) elongated flexible arms, and eight (8) elongated flexible arms.

### Configurations #2 and #3: Locking by Snap-Fit/Bayonet, Releasing by Bayonet

The distal end of the plunger rod may include an entrance tip that includes a number N of fins that are angularly distributed around and radially extend outward from a longitudinal axis of the plunger rod, and a segmented (discrete, discontinued) lock groove that is circumferentially formed in the distal end between the N fins and the rest of the plunger rod. The plunger engagement member (PEM) includes a bored cylindrical member with an inner wall, and a number N of ribs that radially extend inward from the inner wall toward a longitudinal axis of the bored cylindrical member and configured to respectively lock the N fins. The PEM also includes a number S of bayonet slots, or grooves, that are circumferentially formed in the bored cylindrical member. The S bayonet slots are designed as three-dimensional curves (helixes) that encircle the longitudinal axis of the bored cylindrical member at a constant radial distance from the longitudinal axis. The helical slots are designed such that they enable rotational motion of the PEM up to a rotational angle that is required to lock the fins in the PEM's ribs and/or (depending on the configuration) to unlock the fins from the PEM's ribs.

The PRM includes a hollow cylindrical body through which the plunger rod is movable, and a number S of bayonet pins, or other means with suitable geometry, that radially extend inward from an inner wall of the hollow cylindrical body. The S bayonet pins are respectively engageable with the S bayonet slots of the PEM in a way that the PEM is rotated relative to the stationary PRM.

The PEM is rotatable relative to the PRM between a lock angular position, in which the N fins are respectively aligned with, and consequently locked by, the N ribs to enable retraction of the plunger head by the plunger rod, and a release angular position, in which the N fins are respectively misaligned with the N ribs to enable releasing the plunger rod from the plunger head when the PEM is linearly retracted by the plunger rod into the rest (stow) position in the PRM. Each of the S bayonet slots is designed to enable the rotation of the PEM from the lock angular position to the release angular position when the PEM is linearly moved into the rest (stow) position in the PRM, and from the release angular position to the lock angular position when the PEM is linearly moved away from the rest position in the PRM.

Rotating the PEM from the lock angular position to the release angular position may include rotating the PEM clockwise (or counterclockwise) by a release angle (γ), and rotating the PEM from the release angular position to the lock angular position may include rotating the PEM counterclockwise (or clockwise) by a lock angle (γ), wherein the value of the release/lock angle (γ) is γ=180°/N. For example, for N=4 the value of the lock angle is γ=180°/4=45°. The value of N may be selected from the group consisting of: N=2, N=3, N=4, N=5, and N=6 (where N is the number of fins in the plunger rod and ribs in the PEM).

The distal end of the plunger rod may also include a pushing flange. The pushing flange may have a diameter larger than an inner diameter of the bored cylindrical member of the PEM to enable pushing the PEM in unison with the plunger rod in the forward direction (i.e., in the reservoir emptying direction) as the plunger rod is moved forward in the disposable reservoir. The N fins of the plunger rod respectively include N pull ledges and when the PEM is in the lock angular position, the PEM is sandwiched between the pushing flange and the N pull ledges and moveable in unison with the plunger rod in a backward direction (in the reservoir refilling direction) as the plunger rod is retracted (drawn back) in the disposable reservoir. The entrance tip of the plunger rod may be shaped as a segmented hemispherical tip, segmented conical tip, or as a cruciform Phillips screwdriver head.

### Configuration #2: Connection by Snap-Fit, Disconnection by Bayonet

In some embodiments the plunger rod is lockable in the PEM by using a snap-fit connection means and releasable from the PEM by using a bayonet connection means. In these embodiments, the PEM provides both a snap-fit means for locking the plunger rod in the PEM, and a bayonet means to facilitate releasing of the plunger rod from the PEM. The snap-fit connection means may include the distal end of the plunger rod and a snap-fit feature of the PEM, and the bayonet release means may include a bayonet feature of the same PEM, and the PRM. The snap-fit connection in these embodiments is also a non-reversible snap-fit connection, meaning that once the plunger rod is locked in/by the PEM by snap-fitting it to the PEM, the plunger rod can be released from the PEM only by using the PRM.

The PEM may initially be positioned at the distal end of the disposable reservoir, and the plunger rod may be lockable in/by the PEM in the lock angular position by moving the plunger rod forward in the disposable reservoir, through the PRM, until the N fins of the plunger rod are respectively snap fitted by the N ribs of the PEM. When the plunger rod is locked in/by the PEM, it can be released from the PEM by retracting the PEM to the rest (stow) position in the PRM to thereby impart a twisting motion to the PEM relative to the PRM, from the lock angular position to the release angular position, to thereby release the plunger rod from the PEM.

To facilitate snap-fitting of the N ribs of the PEM into the segmented lock groove in the distal end of the plunger rod the bored cylindrical member of the PEM may be reversibly expandable radially outwardly by the N fins of the plunger rod pushing the N rigid ribs, or the bored cylindrical member of the PEM may be rigid (non-expandable) and the N ribs reversibly compressible by N fins, or the N ribs of the PEM may be reversibly compressible by the N fins of the plunger rod and the bored cylindrical member of the PEM may be reversibly expandable radially outwardly as well.

### Configuration #3: Connection & Disconnection by Bayonet

In other embodiments the PEM may initially be stowed in the PRM in the release angular position, in which case the plunger rod may be locked in/by the PEM by linearly moving the plunger rod forward through the PRM such that the plunger rod linearly moves the PEM away from the stow position in the PRM while simultaneously imparting a twisting motion to the PEM relative to the PRM that causes the PEM to rotate from the release angular position to the lock angular position. When the plunger rod is locked in/by the PEM, it can be released from the PEM by retracting the PEM back to the rest (stow) position in the PRM to thereby impart a twisting motion to the PEM relative to the PRM that causes the PEM to rotate from the lock angular position to the release angular position. The bayonet connection in these embodiments is also a non-reversible connection, meaning that once the plunger rod is locked in/by the PEM, the plunger rod can be released from the PEM only by using the PRM.

According to another aspect of the present invention, there are provided a disposable reservoir with a plunger engagement member (PEM) and a plunger release member (PRM) as shown in the pertinent drawings and described herein, and, in addition, a plunger rod that is engageable with the PEM and releasable from the PEM by the PRM, as shown in the pertinent drawings and described herein.

### a Disposable Reservoir Including a PEM and a PRM (Configurations #1, #2 and #3)

Also provided is a disposable reservoir that is releasably couplable to a reusable part of a drug delivery device, wherein the disposable reservoir includes a plunger head. The plunger head including a plunger engagement member (PEM) and bidirectionally moveable in unison with the PEM between a proximal end of the disposable reservoir and a distal end of the disposable reservoir, and the PEM is configured to engage a plunger rod of the reusable part of the drug delivery device. The disposable reservoir further includes a plunger release member (PRM) that is configured to release the plunger rod from the PEM upon retraction of the PEM to a stow position in the PRM.

The plunger rod is configured to be lockable by or in the PEM by moving the plunger rod forward in the disposable reservoir, through the PRM, until the plunger rod is snap-fitted by the PEM, and the plunger rod is releasable from the PEM by retracting the PEM to the stow position in which the PRM deflects (snap out of) the PEM to release the plunger rod from the PEM.

The PEM may initially be positioned at the distal end of the disposable reservoir and the plunger rod initially lockable by the PEM in a lock angular position by moving the plunger rod forward in the disposable reservoir, through the PRM, until the plunger rod is snap-fitted by the PEM. The plunger rod may be released from the PEM by retracting the PEM to the stow position in the PRM to cause a twisting motion of the PEM, relative to the PRM, from the lock angular position to a release angular position.

The PEM may initially be stowed in the PRM in a release angular position, and the plunger rod may be locked by the PEM by moving the plunger rod forward through the PRM such that the plunger rod linearly moves the PEM away from the stow position in the PRM while simultaneously causing the PEM to rotate from the release angular position to the lock angular position. The plunger rod may be released from the PEM by retracting the PEM back to the stow position in the PRM to rotate the PEM from the lock angular position back to the release angular position.

### BRIEF DESCRIPTION OF THE DRAWINGS

Various exemplary embodiments and aspects are illustrated in the accompanying figures with the intent that these examples be not restrictive. It will be appreciated that for simplicity and clarity of the illustration, elements shown in the figures referenced below are not necessarily drawn to scale. Also, where considered appropriate, reference numerals that are repeated among the figures indicate like, corresponding or analogous elements. Of the accompanying figures:

FIGS. 1A-1C show a snap-fit locking of a plunger rod (150) in a plunger engagement member (140) according to an example embodiment;

FIGS. 1D-1G show a plunger head (130) according to an example embodiment;

FIGS. 2A-2D show a plunger rod (150) and a plunger engagement member (140) as part of a plunger head (130), according to an example embodiment;

FIGS. 2E-2F show an example 4-arm plunger engagement member (140), according to an example embodiment;

FIGS. 2G-2I show an example 6-arm plunger engagement member (140), according to an example embodiment;

FIGS. 3A-3C show an example plunger release member (190) according to an example embodiment;

FIG. 4 shows the position of the plunger engagement (140) relative to the plunger release member (190);

FIGS. 5A-5B demonstrate snap-fit unlocking of a plunger rod (150) from a plunger engagement member (140) by using the plunger release member (190), according to an example embodiment;

FIGS. 6A-6B demonstrate unlocking of a plunger rod (150) from a plunger engagement member (140) when the disposable part of the pump device is coupled (attached) to the reusable part of the pump device;

FIG. 7 shows an example plunger rod (700) in accordance with some embodiments;

FIGS. 8A-8B show an example plunger engagement member (810) in accordance with some embodiments;

FIGS. 9A-9B show an example plunger release member (900) in accordance with some embodiments;

FIGS. 10A-10B show the plunger rod (700) in the plunger engagement member (810) when plunger engagement member (810) is in a stow position in plunger release member (900), according to some embodiments;

FIG. 10C shows the plunger rod (700) locked in the plunger engagement member (810) when the plunger engagement member (810) is away from the stow position in plunger release member (900), according to some embodiments;

FIGS. 10D-10E respectively schematically illustrate a plunger engagement member (1016) in an unlock (release) angular position relative to fins (1070) of a plunger rod, and in a lock angular position relative to the fins (1070) of the plunger rod, according to an example embodiment;

FIGS. 11A-11I show various stages of operation of a pump device, including coupling of a disposable part (1100) of the pump device to a reusable part (1110) of the pump device and decoupling of the disposable part (1100) from the reusable part (1110), according to an example embodiment;

FIGS. 12A-12M show various stages of operation of a pump device, including coupling of a disposable part (1200) of the pump device to a reusable part (1210) of the pump device and decoupling of the disposable part (1200) from the reusable part (1210), according to example embodiment; and

FIGS. 13A-13F show various positions of a plunger engagement member (1310) in a plunger release member (1330) according to an example embodiment.

### DETAILED DESCRIPTION OF THE INVENTION

The description that follows provides various details of example embodiments. However, this description is not intended to limit the scope of the claims but instead to explain various principles of the invention and exemplary manners of practicing it.

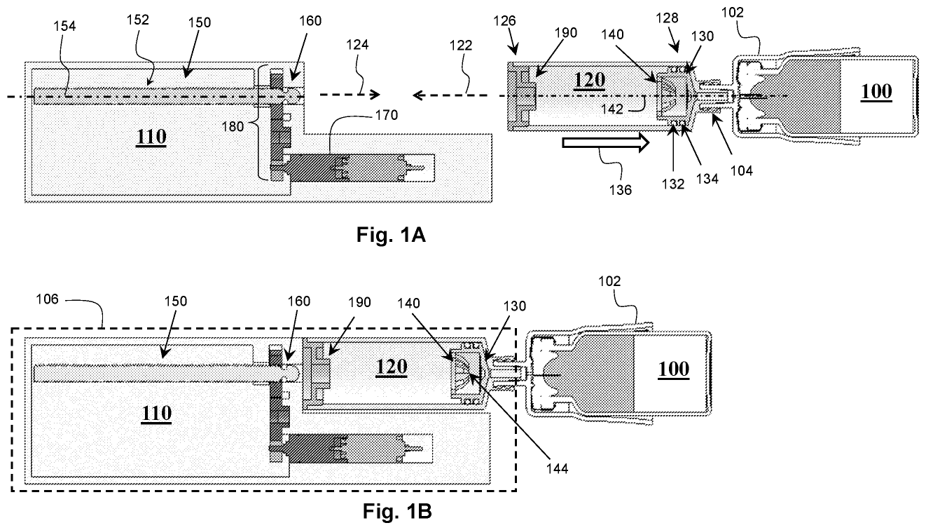

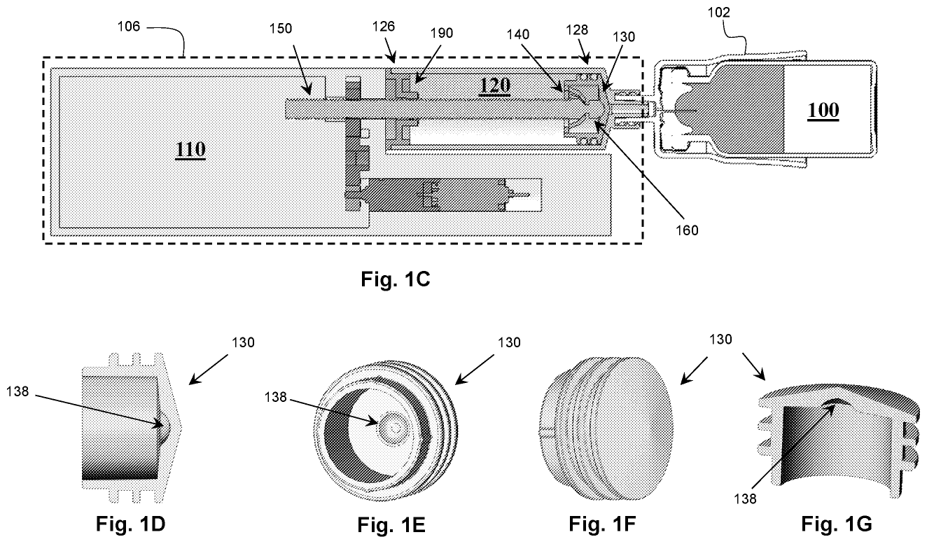

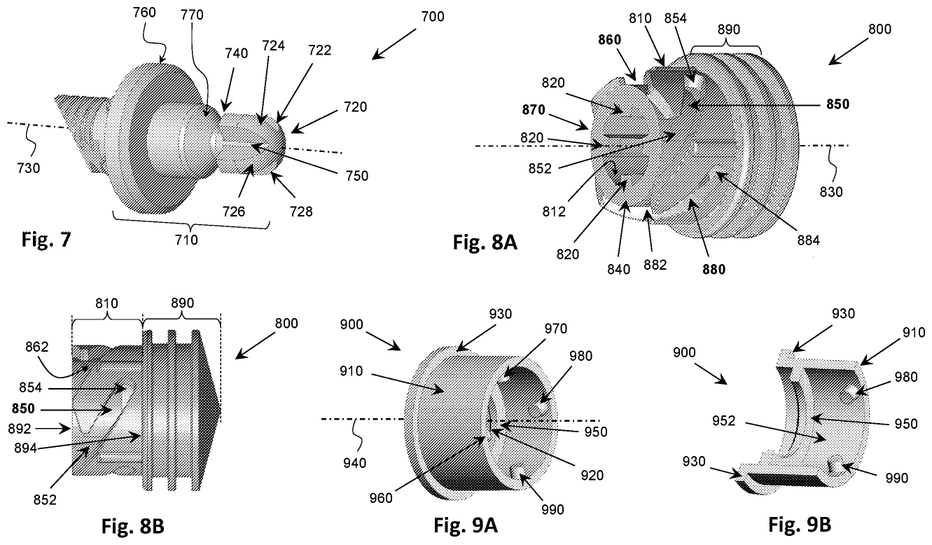

The detailed description and pertinent drawings generally disclose three example configurations:
(1) Configuration #1, which includes a snap-fit connection mechanism for both locking a plunger rod of a pump device in, or by, a plunger head of a pump device and releasing the plunger rod from the plunger head (see, for example, FIGS. 1A-1G, 2A-2I, 3A-3C, 4, 5A-5B, and 6A-6B);
(2) Configuration #2, which, similarly to configuration #1, includes a snap-fit connection feature for locking a plunger rod of a pump device in, or by, a plunger head of the pump device, and a bayonet mechanism for releasing the plunger rod from the plunger head (see, for example, FIGS. 7, 8A-8B, 9A-9B, 10A-10C, and 11A-11I), and
(3) Configuration #3, which includes a bayonet mechanism for both locking a plunger rod of a pump device in, or by, a plunger head of the pump device and releasing (unlocking) the plunger rod from the plunger head (see, for example, FIGS. 7, 8A-8B, 9A-9B, 10A-10C, 12A-12M, and 13A-13F).

Common to the three configurations is the addition of a plunger engagement member (PEM) to the plunger head of the pump device that is designed to engage and lock a distal end of the plunger rod of the pump device, and, in addition, a stationary plunger release member (PRM) that is designed to unlock the plunger rod's distal end from the plunger head. Common to configurations #2 and #3 is a bayonet mechanism that is used to rotate the PEM relative to the PRM, for example to unlock a plunger rod from a plunger head (per configuration #2), and for both locking and unlocking the plunger rod from the plunger head (per configuration #3). The bayonet mechanism used herein is not used in a conventional manner, meaning that the bayonet pins and slots are not used as a locking means but, rather, they are used only to rotate the PEM relative to the stationary PRM, whether from an ‘unlock’ angular position to a ‘lock’ angular position and back to the unlock angular position (as described in connection with configuration #3), or just from a lock angular position to an unlock angular position (as described in connection with configuration #2).

FIGS. 1A-1C schematically illustrate a cross-sectional view of a drug delivery device 106 (see FIG. 1C) according to an example embodiment. The drug delivery device includes a reusable part 110 and a disposable medicament reservoir 120. Reusable part 110 and disposable medicament reservoir 120 are releasably couplable (122, 124) to one another, for example by using a magnet (i.e., using magnetic attraction force), a snap-fit connector, a bayonet connector, etc. For example, a permanent magnet may be fixedly embedded in reusable part 110 or in disposable medicament reservoir 120, and a metal plate magnetically attractable to the magnet may be fixedly embedded in the other part, such that when reusable part 110 and disposable medicament reservoir 120 are brought into proximity with one another, the permanent magnet would magnetically attract the metal plate.

Disposable reservoir 120 includes a plunger head 130 that is bidirectionally slidable along the axial direction of reservoir 120. Plunger head 130 includes a plunger engagement member (PEM) 140. Reusable part 110 includes a plunger rod 150. Plunger rod 150 includes a threaded section 152 and a distal end 160 which is not threaded. Distal end 160 (of plunger rod 150) and plunger engagement member 140 are releasably engageable. Disposable reservoir 120 also includes a bottom cover 190 with a centered opening through which plunger rod 150 is bidirectionally moveable. (Bottom cover 190 is an axisymmetric object that functions as a plunger release member—PRM.) As shown in FIGS. 1A-1C, PRM 190 of disposable reservoir 120 and PEM 140 are separate elements—PRM 190 is affixed (fixedly mounted) to proximal end 126 of reservoir 120, and PEM 140 is bidirectionally slidable (moveable by plunger rod 150 when it is coupled with PEM 140) in reservoir 120 between proximal end 126 and distal end 128 of reservoir 120.

Reusable part 110 includes an electric motor based driving system that includes an electric motor 170 and a gear system 180 that is driven, or powered, by electric motor 170. Gear system 180 may include one or more gears and a nut. The nut is axially held in place and includes an external threading, or profile, that couples with a gear of gear system 180. The nut's internal threading mates the threaded section 152 of plunger rod 150. The configuration of gear system 180 is such that when the nut is rotated by the gear, the nut linearly moves plunger rod 150 through a concentric bore in PRM 190 without rotating plunger rod 150. (The same gear/nut configuration applies to all plunger rods that operate according to any of the configurations that are disclosed herein, for example to plunger rods 700, 1112, 1212, and 1300.) Using gear system 180, electric motor 170 can linearly and bidirectionally move plunger rod 150 in the plunger rod's axial direction, i.e., along longitudinal axis 154 of plunger rod 150. Plunger rod 150 and plunger head 130 are engageable via PEM 140 to enable them to bidirectionally move in unison in disposable reservoir 120 when, and while, disposable reservoir 120 and reusable part 110 are coupled to each other, and plunger rod 150 and plunger head 130 are releasable from one another to enable decoupling of disposable reservoir 120 from reusable part 110.

Disposable reservoir 120 may be filled with liquid drug from vial 100. Vial 100 rests in vial adapter 102, and vial adapter 102 is releasably connectable to disposable reservoir 120 by a Luer-type connector 104. Two sealing O-rings 132 and 134 prevent leakage of liquid drug from reservoir 120.

FIG. 1A shows reusable part 110 and disposable reservoir 120 before they are coupled to one another, plunger rod 150 that is stowed in (fully retracted into) reusable part 110 in a ‘standby’ position, and plunger head 130 that is also in a ‘standby’ (i.e., reservoir prefill) position in which plunger head 130 is positioned at distal end 128 of reservoir 120. In FIG. 1B, reusable part 110 and disposable reservoir 120 are coupled to one another but plunger rod 150 is still stowed in (fully retracted into) reusable part 110 in the ‘standby’ position and plunger head 130 is also in its ‘standby’ position. In FIG. 1C, drug delivery device 106 is in a ‘ready’ position as plunger rod 150 is fully extended from reusable part 110 and engaged with PEM 140 at distal end 128 of reservoir 120. When drug delivery device 106 is in the ‘ready’ position, plunger rod 150, hence plunger head 130, may be retracted rearwardly (e.g., by electric motor 170) to fill reservoir 120 up with medicament, and then, forward again to empty reservoir 120 during medical treatment. (“Rearwardly” means moving plunger head 130 in a direction from distal end 128 to proximal end 126, that is, in the opposite direction of direction 136.)

In general, plunger rod 150 and PEM 140 may be releasably engageable by using, for example, a snap-fit connection mechanism, or a bayonet connection mechanism, or a combination of snap-fit connection and bayonet connection. The snap-fit connection mechanism may be or include, for example, an annular snap-fit connection feature (including a snap-fit ‘lip’, or a segmented lip), or a cantilever snap-fit connection feature, or a combination of annular snap-fit connection feature and cantilever snap-fit connection feature.

### Configuration #1: Locking and Unlocking a Plunger Rod by Snap-Fitting

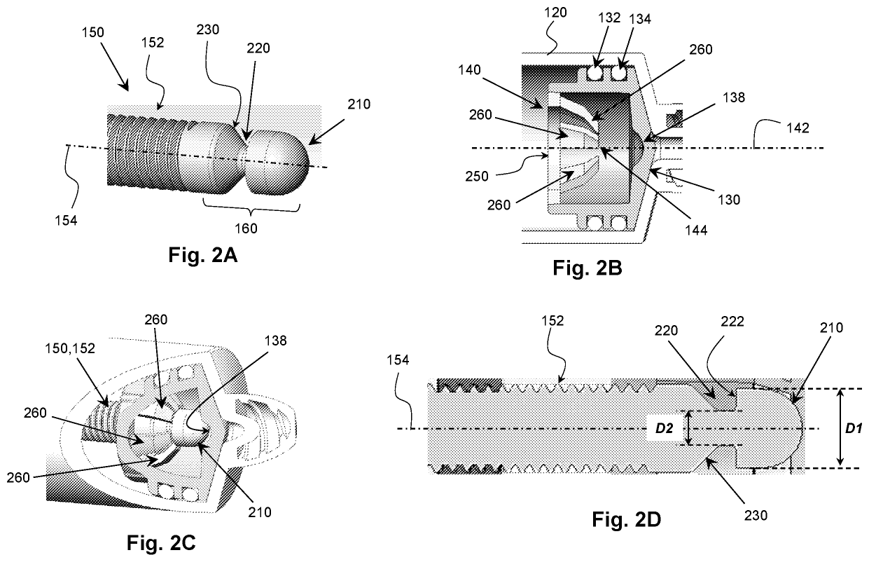

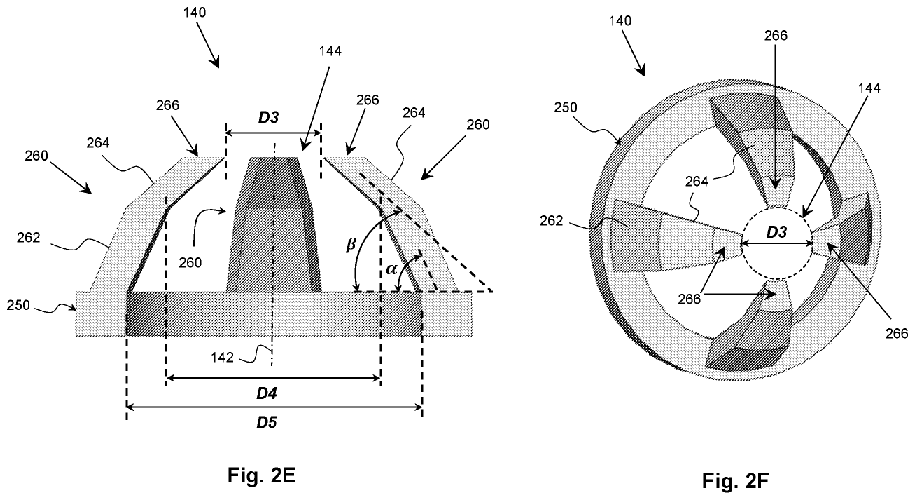

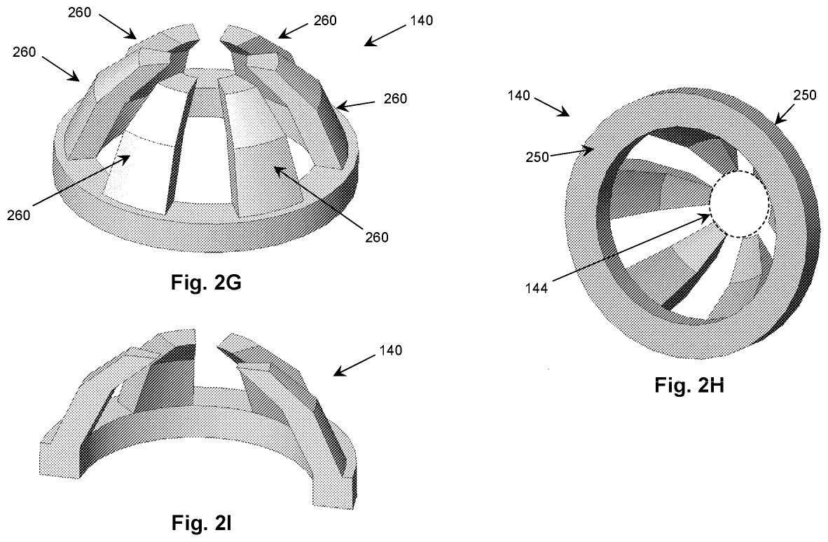

Referring to FIGS. 1A-1G, the snap-fit connection includes distal end 160 of plunger rod 150 and plunger engagement member (PEM) 140. FIGS. 1D-1G show plunger head 130 without a PEM and without O-rings 132 and 134. FIGS. 1D-1E and FIG. 1G show an inner receiving (‘pushing’) concave surface 138 (also shown in FIGS. 2B-2C) of plunger head 130.

Referring also to FIG. 2A, distal end 160 of plunger rod 150 includes an entrance tip 210 and an annular lock groove 220 that is circumferentially formed in distal end 160 between entrance tip 210 and the rest of plunger rod 150. Entrance tip 210 may have a convex shape, for example hemispherical shape (as shown in FIG. 2A, 2C, 2D), or other shapes. For example, entrance tip 210 may be conically shaped, or it may be a truncated cone, or a pyramid, or a frustum pyramid, etc.

FIG. 2B shows PEM 140 before it snaps (snap-fitted) into annular lock groove 220 of plunger rod 150. FIG. 2C is a 3D cut away view showing PEM 140 snapped into place (i.e., into annular lock groove 220) in plunger rod 150. Referring to FIG. 2B, PEM 140 includes a ring-shaped base 250 and multiple equally elongated and flexible arms 260. Multiple elongated flexible arms 260 circularly extend from ring-shaped base 250 and may be angularly equidistant from one another. Elongated flexible arms 260 extend from ring-shaped base 250 at an acute angle and bend inwardly (converge) towards a central axis 142 of plunger engagement member 140. This way, multiple elongated flexible arms 260 form a flexible cap-like structure (as shown, for example, in FIGS. 2F-2H) with a central opening 144 at its apex that is defined by a snap-fit lip that is snap-fittable into annular lock groove 220. Plunger engagement member 140 is concentrically fixedly embedded in plunger head 130 so that the two objects are moveable in unison in disposable reservoir 120 in the reservoir's emptying direction (direction 124) as well as in the opposite direction (i.e., in the reservoir's filling direction, which is the direction opposite direction 124). FIG. 2C shows plunger rod 150 snap-fitted by PEM 140.

FIG. 2E shows a cross-sectional view of a 4-arm PEM 140, and FIG. 2F shows a three-dimensional view of the 4-arm PEM 140. (As shown in other drawings and described herein, PEM 140 may include a different number of elongated flexible arms.) Referring to FIG. 2E, each elongated flexible arm 260 includes a proximal section 262 that extends from ring-shaped base 250 at an acute angle α relative to ring-shaped base 250 (where 0°<α<90°, for example α=60°). Each elongated flexible arm 260 also includes a distal section 264 that contiguously extends from the proximal section at a smaller acute angle β relative to ring-shaped base 250 (i.e., 0°<β<α, for example β=45°). The distal sections (264) of multiple elongated flexible arms 260, or the entire flexible arms, are configured (e.g., sized, shaped and made of material(s)) in a way that enables the flexible arms to lock into annular lock groove 220 of plunger rod 150, and to be retracted by plunger rod 150 towards proximal end 126 of disposable reservoir 120. Each elongated flexible arm 260 also includes a pull, or contact, surface 266. Each pull surface 266 may be perpendicular to central axis 142 of plunger engagement member 140 to maximize pull contact surface(s) between pull surfaces 266 and an annular pull ledge (‘shoulder’) 222 of plunger rod 150 (see FIG. 2D).

When plunger rod 150 is retracted (e.g., pulled back) in reservoir 120, pull surfaces 266 (a pull surface 266 for each flexible arm 260) enable annular pull ledge 222 of plunger rod 150 to move plunger engagement member 140, hence plunger head 130, backward (rearward) in reservoir 120 in unison with the movement of plunger rod 150 while elongated flexible arms 260 snap (snap-fitted) into annular lock groove 220. Plunger rod 150 may controllably be retracted in reservoir 120, for example to fill an empty reservoir 120 with drug, or for releasing plunger rod 150 from plunger head 130 in preparation for decoupling of disposable reservoir 120 from reusable part 110. Annular pull ledge 222 of plunger rod 150 pulls PEM 140 in unison with the movement of plunger rod 150 in the backward (filling) direction by causing annular pull ledge 222 to contact pull surfaces 266 of elongated flexible arms 260 and then, pulling distal sections 264 of elongated flexible arms 260 by plunger rod 150 while plunger rod 150 is retracted (moved rearwardly) in reservoir 120.

Annular lock groove 220 may also include a conical surface 230 (FIGS. 2A and 2D). depending on the design specifics of PEM 140 and plunger 150, conical surface 230 may enable further movement of distal end 160 of plunger rod 150 through central opening 144 in the flexible cap-like in a forward (emptying) direction 136 (FIG. 1A) until entrance tip 210 of plunger rod 150 contacts (sits or received in) an inner receiving (‘pushing’) concave surface 138 (FIG. 1D, FIGS. 2B-2C) of plunger head 130. Contacting receiving concave surface 138 of plunger head 130 by plunger rod 150 enables plunger rod 150 to push plunger head 130 in the forward (emptying) direction, i.e., in direction 136 (FIG. 1A). That is, when the flexible cap-like structure of PEM 140 snaps (is snap-fitted) into annular lock groove 220 of plunger rod 150, entrance tip 210 of plunger rod 150 can push receiving concave surface 138 of plunger head 130 to slide plunger head 130 forward (in emptying direction 136) to expel medicament out of the reservoir. Receiving concave surface 138 is centered in plunger head 130 (i.e., concave surface 138 lies on axis 142, see FIG. 2B) and, in addition, the concavity profile of receiving concave surface 138 rigidly (or elastically) matches the convexity profile of entrance tip 210 of plunger rod 150. The two mechanical features (i.e., centered concave surface 138 and concavity-convexity complementary profiles) are useful in avoiding uneven mechanical stresses (e.g., bending, compression and/or shear stresses) from developing in plunger head 130, in PEM 140, or in plunger rod 150 when plunger rod 150 extends, i.e., moves in emptying direction 136 (FIG. 1A).

Elongated flexible arms 260 are reversibly deflectable radially outward by entrance tip 210 of plunger rod 150 as entrance tip 210 is moved forward (in the emptying direction 136, FIG. 1A) through central opening 144 (FIGS. 1B, 2B, 2E, 2F) of the flexible cap-like structure, and snappable (snap-fittable) into annular lock groove 220 of plunger rod 150 as entrance tip 210 is moved passed central opening 144 of the flexible cap-like structure in a way that prevents release of plunger rod 150 from PEM 140 when plunger rod 150 is retracted (moves rearwardly) in reservoir 120. To impart this capability to PEM 140, the diameter D3 (see, for example, FIGS. 2E, 2F) of opening 144 in PEM 140 at rest (i.e., when flexible arms 260 are not deflected but, rather, are in their rest, or stress-free, position) is smaller than the diameter D1 (see FIG. 2D) of end point 210 and similar to, or a greater than, the diameter D2 (FIG. 2D) of the ‘neck’ of annular lock groove 220. So, to enable plunger rod 150 to retract PEM 140 (hence plunger head 130) when plunger rod 150 is retracted backwards in reservoir 120, the value of diameter D3 satisfies the condition D3<D1. (The value of diameter D3 may additionally satisfy the condition D32 D2 but it may also be slightly smaller than diameter D2.)

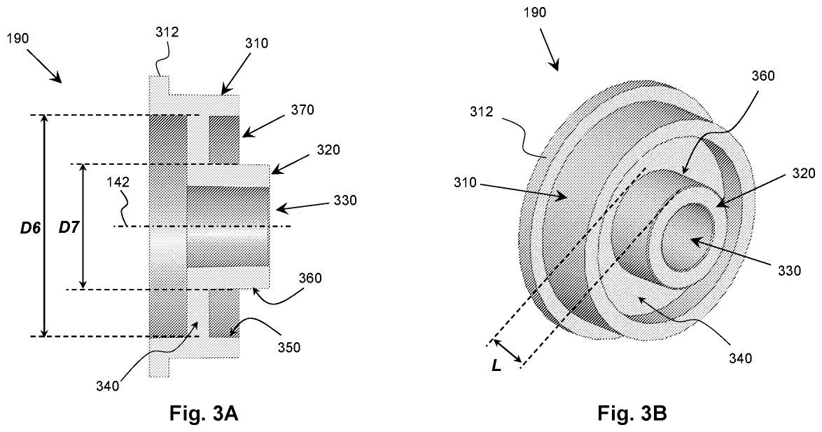

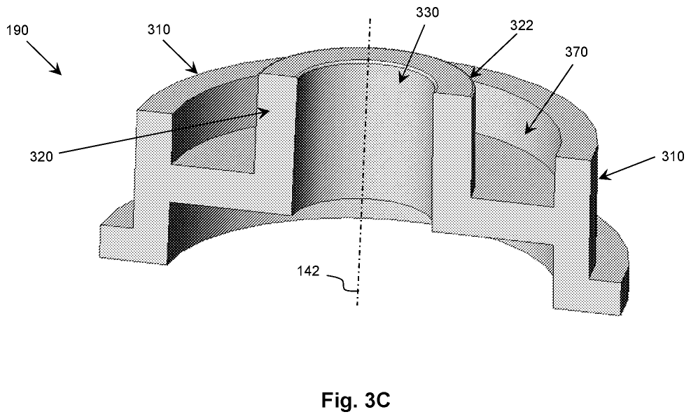

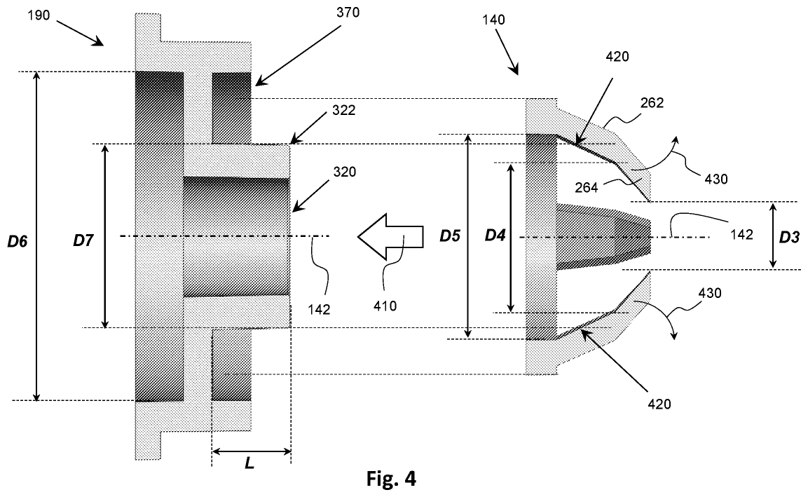

Entrance tip 210 of plunger rod 150 includes a curved ‘entrance side’ that enables entrance tip 210 to deflect elongated flexible arms 260 radially outward, away from central axis 142 (FIGS. 1A, 2B) of PEM 140. Diameters D4 and D5 (FIG. 2E) in PEM 140 facilitate releasing of plunger rod 150 from PEM 140, as described further below, for example in connection with FIGS. 3A-3B, FIG. 4, and FIGS. 5A-5B. Briefly, to deflect flexible arms 260 radially outward the external diameter D7 (FIG. 3A, FIG. 4) of concentric cylindrical body 320 is smaller than the inner diameter D5 of ring-shaped base 250 of PEM 140, and preferably greater than diameter D4 so that elongated flexible arms 260 can be deflected by the cylindrical body 320 pushing proximal section 262 sideways. If the external diameter D7 of cylindrical body 320 is smaller than diameter D4, elongated flexible arms 260 can be deflected by cylindrical body 320 pushing distal section 264 sideways.

The number of elongated flexible arms 260 that PEM 140 may include may be 2n (where n=1, 2, 3, . . . etc.). Elongated flexible arms 260 are equally sized and shaped, and they may be angularly equidistant from one another on ring-shaped base 250. Plunger engagement member (PEM) 140 may include, for example, four flexible arms, six flexible arms, eight flexible arms, etc. (Other numbers of flexible arms may be used. For example, PEM 140 may include an odd number of elongated flexible arms, for example three flexible arms, five flexible arms, etc.)

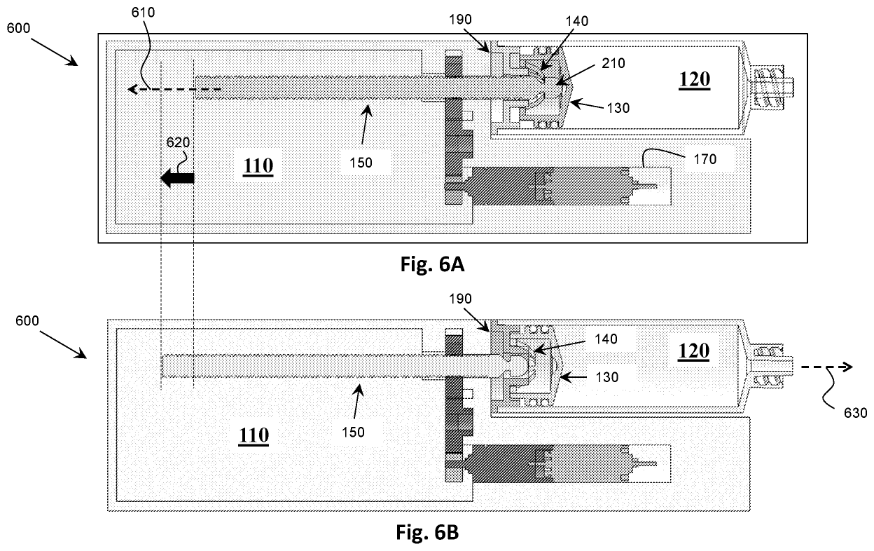

Looking at FIG. 2C (for example), the snap fit connection mechanism that includes PEM 140 and distal end 160 of plunger rod 150 is non-reversible in the sense that distal end 160 is not provided with a ‘retraction side’ that would enable it to disengage (release itself) from PEM 140 ‘on its own’, i.e., by sliding ‘itself’ out of PEM 140. (Had distal end 160 of plunger rod 150 included a retraction side, plunger rod 150 would not have been able to pull PEM 140, hence plunger head 130, backward to fill reservoir 120 up with drug.) Plunger rod 150 is, nevertheless, releasable from PEM 140 by using plunger release member (PRM) 190 of disposable reservoir 120 as an auxiliary (ejection) member. (PRM 190 is mechanically separated from, and functions independently of, PEM 140.) The snap-fit release functionality needed to release (disengage) plunge rod 150 from PEM 140 is imparted to PRM 190 in the way shown in FIGS. 3A-3C, which are described below. (PRM 190 (a ‘snap-out’ element, or ring) is shown in FIGS. 1A-1C, FIGS. 3A-3B, FIG. 4, FIGS. 5A-5B, and in FIGS. 6A-6B.)

FIGS. 2E and 2F depict an example plunger engagement member (PEM) that includes four elongated flexible arms 260. FIGS. 2G-2I depict a PEM that includes six elongated flexible arms 260. However, numbers of elongated flexible arms other than four or six may be used. The number of flexible arms of a PEM may depend on one or more of the following parameters:
(1) an electrical parameter of the pump device;
(2) an electrical parameter of the power source (e.g., battery) that powers the pump device;
(3) a mechanical parameter of the flexible arms, and
(4) a mechanical friction between the plunger head and the cylindrical body of the disposable reservoir in which the plunger head moves.

An electrical parameter of the pump device may be, for example, the electrical energy that the motor (e.g., motor 170, FIG. 1A) requires to drive the plunger rod (e.g., plunger rod 150), for example back and forth, a predetermined number of times. An electrical parameter of the power source may be, for example, the total electrical energy capacity of the power source, or battery efficiency. A mechanical parameter of the flexible arms may be, or may relate to, the flexible arms' ability to deflect (flex) radially outward to a desired distance at least a desired number of times without losing their flexing capability. Additionally, or alternatively, a mechanical parameter of the flexible arms may be, or may relate to, the ease of flexing of the flexible arms. Additionally, or alternatively, a mechanical parameter of the flexible arms may be, or may relate to, the size, shape and material of the flexible arms. By way of example, the stiffer the flexible arms, the smaller the number of flexible arms that may be used to avoid overburdening the motor. The easier the flexible arms flex (deflect), the greater their number can be. In another example, the greater the electric capacity of the power source (e.g., battery), the greater the number of flexible arms that can be used, etc.

FIG. 3A shows a cross-sectional view of PRM 190, and FIG. 3B shows a three-dimensional view of PRM 190. PRM 190 includes an external cylindrical body 310 and a hollow cylindrical body 320. Cylindrical body 310 has an internal diameter D6, and cylindrical body 320 has an external diameter D7, where D7<D6. Cylindrical body 320 includes a bore 330 through which plunger rod 150 can freely move. Cylindrical body 320 also includes a flange 312. Flange 312 projects outwardly from, and encircles, cylindrical body 310 to form therewith an “L”-shaped object around axis 142 that enables affixing PRM 190 to proximal end 126 of reservoir 120. Cylindrical bodies 310 and 320 are interconnected by an annular base 340. Annular base 340 projects outwardly from, and encircles, cylindrical body 320 to form therewith an “L”-shaped object around axis 142. Inner wall 350 of cylindrical body 310, external wall 360 of cylindrical body 320 and annular base 340 define an open annular channel (“OAC”) 370 around cylindrical body 320 and axis 142. Open annular channel (OAC) 370 provides a docking station that is configured for receiving (stowing) PEM 140 during and after disengagement of plunger rod 150 from PEM 140.

Cylindrical body 320 is configured to deflect elongated flexible arms 260 radially outward to enable releasing plunger rod 150 from the flexible cap-like structure of PEM 140 when ring-shaped base 250 of PEM 140 is moved into the rest (stow) position in OAC 370 when motor 170 (FIG. 1A) fully retracts plunger rod 150 in reservoir 120. Open annular channel 370 is configured to receive (accommodate) part of plunger head 130, ring-shaped base 250 of PEM 140, and part of elongated flexible arms 260. The length L (FIG. 3B) of cylindrical body 320 is a design parameter used to determine the first contact points on flexible arms 260 that concentric cylindrical body 320 of PRM 190 contacts when PEM 140 is moved into the stow position in PRM 190.

Releasing Plunger Rod 150 from PEM 140

As described herein, diameter D1 (FIG. 2D) of entrance tip 210 of plunger rod 150 is greater than diameter D3 (FIGS. 2E-2F) of opening 144 of plunger engagement member (PEM) 140 in a stress-free (non-deflected) state to enable entrance tip 210 of plunger rod 150 to deflect flexible arms 260 of PEM 140 radially outward, after which flexible arms 260 resume (return to) their previous (original, rest, non-deflected) state to snap fit into annular lock groove 220 of plunger rod 150.

PRM 190 is stationary in reservoir 120, whereas PEM 140 is bidirectionally moveable axially (longitudinally) in reservoir 120 between proximal end 126 of the reservoir and distal end 128 of the reservoir. Referring to FIG. 4, when PEM 140 is moved backwards (retracted in direction 410) by plunger rod 150 along axis 142 (which is also the longitudinal axis of reservoir 120), PEM 140 moves towards the stow position in open annular channel (OAC) 370. During movement of PEM 140 towards the stow position in OAC 370 the circular leading edge 322 of cylindrical body 320 contacts the inner walls of the proximal sections 262 of PEM 140 at deflection engagement points (i.e., at a deflection engagement point in each proximal section 262), for example at deflection engagement points 420 in a middle section of proximal sections 262. Moving PEM 140 further in this direction (in direction 410) would cause the circular leading edge 322 (FIGS. 3C and 4) of cylindrical body 320 to deflect proximal section 262 of each flexible arm 260, hence the entire respective elongated flexible arms 260, radially outward. The greater the outward deflection 430 of flexible arms 260, the greater the value of the diameter D3 of opening 144 (FIGS. 2F and 2H). Plunger rod 150 is releasable from PEM 140 when the value of D3 is (gets) greater than D1, where D1 is the diameter of entrance tip 210 of plunger rod 150. Depending on the design specifics of PEM 140 and PRM 190, the maximum radially outward deflection 430 of flexible arms 260 occurs when PEM 140 reside fully or partly (depending on the design specifics) in the stow position in PRM 190. Accordingly, PEM 140 and PRM 190 are designed such that the condition D3>D1 is satisfied when PEM 140 is stowed in PRM 190, namely, when PEM 140 rests in open annular channel (OAC) 370 in PRM 190.

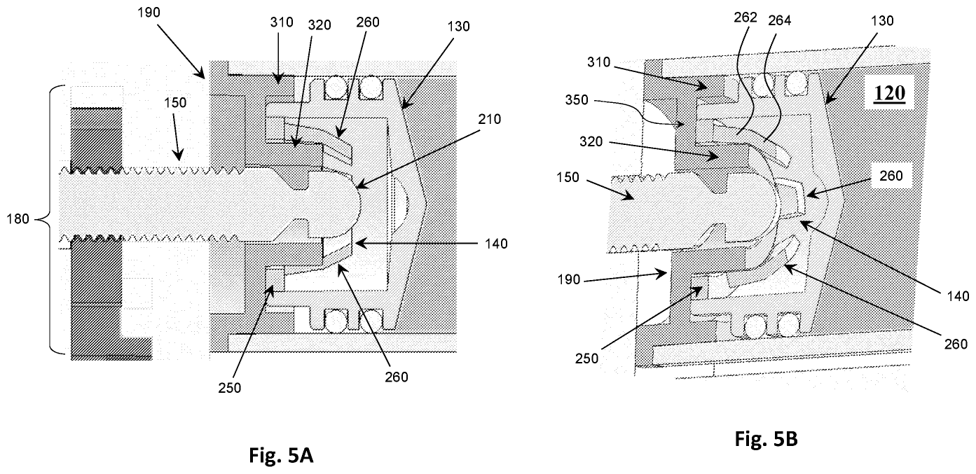

FIG. 5A and FIG. 5B respectively show two-dimensional and three-dimensional cross-sectional views of PEM 140 (and plunger head 130) at the stow position in PRM 190, which serves as the docking station for PEM 140 and plunger head 130. When PEM 140 (jointly with plunger head 130) is at the stow position in PRM 190, the deflection of flexible arms 260 of PEM 140 is maximal (D3>D1) such that plunger rod 150 can be easily released (pulled away, or disconnected) from PEM 140, hence from plunger head 130, for example by motor 170 (FIG. 1A).

FIG. 6A depicts pump device 600 with disposable reservoir 120 coupled to reusable part 110, and plunger engagement member (PEM) 140 is just about to be stowed in plunger releasing member (PRM) 190 (which also functions as a docking station). Referring to FIG. 6A, entrance point 210 of plunger rod 150 is still snaped-fitted by PEM 140, for which reason plunger rod 150 cannot be released yet from PEM 140 by motor 170, and, consequently, disposable reservoir 120 cannot be decoupled from reusable part 110. (To decouple disposable reservoir 120 from reusable part 110, plunger rod 150 must first be released from plunger head 130, i.e., from PEM 140.) Motor 170 may retract plunger rod 150 further in direction 610 by a distance 620 to stow PEM 140 in PRM 190 (docking station), as shown in FIG. 6B.

FIG. 6B depicts pump device 600 with disposable reservoir 120 still coupled to reusable part 110, but with PEM 140 stowed in the docking station (in PRM 190). Referring to FIG. 6B, entrance point 210 of plunger rod 150 is no longer snapped-fitted by PEM 140, so plunger rod 150 can now be released from PEM 140, and disposable reservoir 120 decoupled from reusable part 110, by moving (e.g., manually) disposable reservoir 120 in direction 630, away from reusable part 110. So, stowing PEM 140 in PRM 190 by motor 170 automatically ejects plunger rod 150 from PEM 140. So, PRM 190, by functioning both as an ejection means and as a stowing means, ejects plunger rod 150 from PEM 140 when plunger rod 150 pulls PEM 140 into the stow position in PRM 190. (The ejection of plunger rod 150 from PEM 140 is more clearly shown in FIGS. 5A-5B.)

Features that are Common to Configurations #2 and #3

Configurations #2 and #3 make use of a bayonet connection means. As described herein, the difference between configuration #2 and configuration #3 is that in configuration #2 the plunger rod is lockable (retained) in the plunger engagement member (PEM) by using a snap-fit feature and releasable from the PEM by using a bayonet feature, and in configuration #3 the plunger rod is both lockable (retained) in the PEM and releasable from the PEM by using a bayonet feature.

In some embodiments, the distal end of the plunger rod includes an entrance tip that includes a number N of equidistantly angularly spaced fins that radially extend outward from a longitudinal axis of the plunger rod. The distal end of the plunger rod also includes a segmented circumferential lock groove that is circumferentially formed in the distal end by the N fins, between the N fins and the rest of the plunger rod.

FIG. 7 depicts an example plunger rod 700 according to some embodiments, FIGS. 8A-8B depict an example plunger head 800 that includes an example plunger engagement member (PEM) 810 according to some embodiments, and FIGS. 9A-9B depict an example plunger release member (PRM) 900 according to some embodiments. PEM 810 and PRM 900 jointly make up a mechanism in which PEM 810 is, in general, a cylindrical male object with circumferential bayonet slots, or grooves, and PRM 900 is, in general, a cylindrical female object with internal radial bayonet pins, and the two objects are designed to mate and depart from one another with a twisting motion.

The bayonet slots (or grooves) are circumferentially formed in bored cylindrical member 840 of the PEM 800. The bayonet slots are designed as three-dimensional curves (helixes) that encircle longitudinal axis 830 of the bored cylindrical member at a constant radial distance from longitudinal axis 830. The helical slots (e.g., slot 850) are designed such that a rotational (twist) motion of PEM 800 about its longitudinal axis 830 is enabled by the helical slots up to a rotational angle that is required to lock the fins (e.g., fin 724) in PEM 800 and/or (depending on the configuration) to unlock the fins from the PEM.

Referring to FIG. 8A, PEM 810 may be fixedly embedded in, or attached to, leading section 890 of plunger head 800, or it may be manufactured as part of plunger head 800 (for example by using plastic injection molding), so that the two distinct objects (PEM, plunger head) are moveable in unison in a disposable reservoir in the reservoir's emptying direction as well as in the opposite direction (i.e., in the reservoir's filling direction.

Plunger rod 700, plunger head 800 and PRM 900 can be used in at least the two configurations described herein (i.e., configurations #2 and #3). In configuration #2 plunger rod 700 is engageable with PEM 810 by using a snap-fit connection, and disengageable (releasable) from PEM 810 by using a bayonet mechanism that includes a number S of spiraling bayonet slots and a same number of bayonet radial pins that are insertable into, and slidable in, the bayonet slots. The bayonet slots are designed as three-dimensional curves (helixes) that encircle a longitudinal axis of a bored cylindrical member of the PEM at a constant radial distance from the longitudinal axis. The helical slots of the PEM are designed such that the slots enable the PEM to rotate about its longitudinal axis by a rotational angle that is required to lock the fins in the PEM and/or (depending on the configuration) to unlock the fins from the PEM. In configuration #3 plunger rod 700 is both engageable with (lockable by) PEM 810 by using the bayonet mechanism and disengageable (releasable) from PEM 810 by using the bayonet mechanism.

Referring to FIG. 7, plunger rod 700 includes a distal end 710 including an entrance tip 720. By way of example, entrance tip 720 includes four, equidistantly angularly spaced, fins 722, 724, 726 and 728. (Entrance tip 720 may include any suitable number N of angularly spaced fins.) Fins 722, 724, 726 and 728 extend radially outward (away) from longitudinal axis 730 of plunger rod 700. Distal end 710 of plunger rod 700 also includes a segmented lock groove 740 that is circumferentially formed in distal end 710 between fins 722, 724, 726 and 728 and conical surface 770. Circumferential lock groove 740 is segmented by the fins 722, 724, 726 and 728 being circumferentially interspersed with a plurality of recesses in an alternating manner such that each two adjacent fins are spaced apart (separated) by a recess. For example, fins 724 and 726 are adjacent fins, extend radially outward from longitudinal axis 730, and spaced apart by recess 750. (For comparison, circumferential lock groove 220 in FIGS. 2A and 2D is a continuous groove.)

Fins 722, 724, 726 and 728, and the recesses therebetween, form a cruciform object resembling a Phillips screwdriver head. Entrance tip 720 may generally be shaped as a segmented hemispherical tip, segmented conical tip, or as a cruciform Phillips screwdriver head. Each of example fins 722, 724, 726 and 728 has a curved ‘entrance side’ to impart to the fins the snap-fitting capability that is mentioned herein in connection with configuration #2. In configuration #3, the fins do not need a curved entrance side to engage a PEM or to release the fins from the PEM. Therefore, in configuration #3 each of the fins may have a curved entrance side, though the curved entrance sides would be, in this case, of little or no use because this configuration does not require a snap-fit deflection capability.

Referring to FIGS. 8A-8B, plunger engagement member (PEM) 810 includes a bored cylindrical member 840 with an inner wall 812, and four ribs 820. Ribs 820 radially extend inward from inner wall 812 of PEM 810, towards longitudinal axis 830 of plunger head 800. Ribs 820 are equidistantly angularly spaced, or distributed, around inner wall 812 of PEM 810, about longitudinal axis 830 of plunger head 800 (and cylindrical body 840). Ribs 820 are interspersed with a same number (in this example four) of recesses in an alternating manner such that each two adjacent ribs 820 are spaced apart by a recess. (A PEM may include a number ‘N’ of ribs like ribs 820, and, accordingly, a same number ‘N’ of recesses.)

Ribs 820 of PEM 810 are designed to respectively lock fins 722, 724, 726 and 728 of plunger rod 700. Locking fins 722, 724, 726 and 728 by ribs 820 may be implemented, according to configuration #2, by snap fitting distal end 710 of plunger rod 700 by PEM 810, or, according to configuration #3, by inserting plunger rod 700 into PEM 810 while causing plunger head 800 (hence PEM 810) to rotate, about its longitudinal axis 830, from a ‘release’ position, in which fins 722, 724, 726 and 728 are free to respectively pass through the recesses between ribs 820, to a ‘locking’ position in which ribs 820 of PEM 810 are respectively aligned with fins 722, 724, 726 and 728 to thereby lock, or retain, the fins.

PEM 810 also includes four bayonet slots 850, 860, 870 and 880. Bayonet slots 850, 860, 870 and 880 are formed on the outer periphery of cylindrical body 840, and generally arranged as spiral slots. In the example PEM shown in FIGS. 8A-8B each bayonet slot spirals in counterclockwise direction about longitudinal axis 830 of PEM 810, towards axially leading section 890 of plunger head 800. PEM 810 is an axially trailing section of plunger head 800. (“Leading section 890” is a section of plunger head 800 that is generally forward of tip 720 of plunger rod 700 when plunger rod 700 is coupled with PEM 810 and moves plunger head 800 to expel medicament out of the disposable reservoir. “Trailing section 810” is the PEM section that is generally aft tip 720 of plunger rod 700 when plunger rod 700 is coupled with PEM 810 and moves plunger head 800 in the forward direction to expel medicament out of the disposable reservoir.)

Every bayonet slot has an entrance point at the proximal/trailing end 892 (FIG. 8B) of PEM 810, and an end point at the distal/leading end 894 (FIG. 8B) of PEM 810, and each bayonet slot curves about axis 830 between its entering point and its end point. For example, bayonet slot 850 has an entrance point 852 at the trailing end 892 of PEM 810, and an end point 854 at the leading end 894 of PEM 810. Similarly, bayonet slot 860 has an entrance point 862 (FIG. 8B) at the trailing end 892 of PEM 810, and an end point (not shown in FIGS. 8A-8B) at the leading end 894 of PEM 810. Similarly, bayonet slot 870 has an entrance point (not shown in FIGS. 8A-8B) at the trailing end 892 of PEM 810, and an end point (also not shown in FIGS. 8A-8B) at the leading end 894 of PEM 810. Similarly, bayonet slot 880 has an entrance point 882 at the trailing end 892 of PEM 810, and an end point 884 at the leading end 894 of PEM 810.

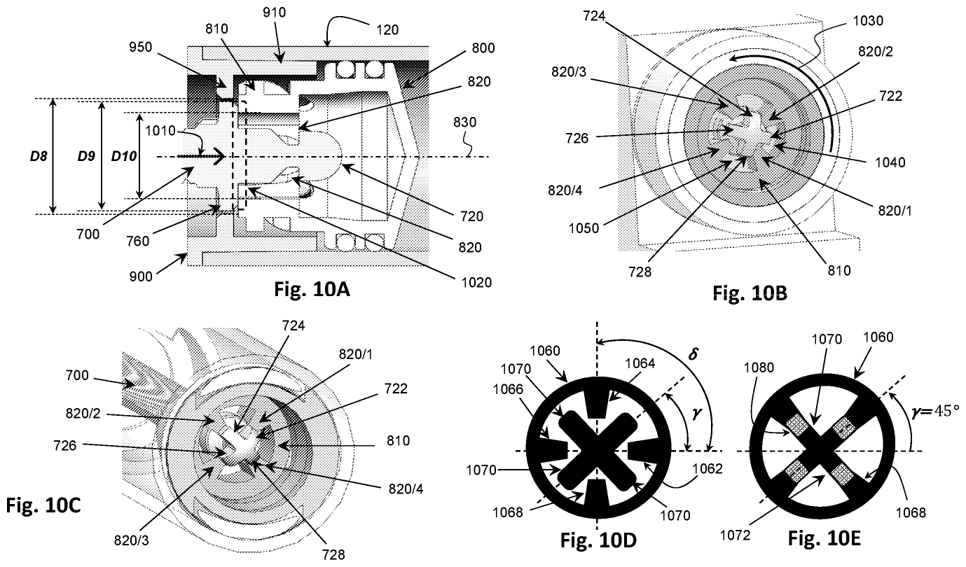

Referring to FIGS. 9A-9B, plunger release member (PRM) 900 includes a hollow cylindrical body 910 with a bore 920. Cylindrical body 910 also includes an external flange (ledge) 930. Ledge 930 circumferentially projects outwardly from, and encircles, cylindrical body 910 to form therewith an “L”-shaped object around longitudinal axis 940 of cylindrical body 910. The “L”-shaped object enables affixing (mounting) PRM 900 to proximal end 126 of reservoir 120. Cylindrical body 910 also includes an inner flange (ledge) 950. Ledge 950 circularly protrudes inwardly from inner wall 952 of cylindrical body 910, towards longitudinal axis 940 of cylindrical body 910. Ledge 950 has a diameter D8 (see FIG. 10A).

Cylindrical body 910 also includes four bayonet radial pins 960, 970, 980 and 990, the number (four) of which correspond to the four bayonet slots 850, 860, 870 and 880. Bayonet pins 960, 970, 980 and 990 are configured to respectively enter bayonet slots 850, 860, 870 and 880 via the entering points of the bayonet slots. For example, bayonet pin 960 may enter bayonet slot 850 via entrance point 852, bayonet pin 970 may enter bayonet slot 860 via entrance point 862, bayonet pin 980 may enter bayonet slot 870 via its entrance point, and bayonet pin 990 may enter bayonet slot 880 via entrance point 882.

A bayonet connector is generally a connector in which one part of the connector can linearly move relative to the other by rotating it relative to the other. To enable this feature, one part of the bayonet connector includes spiral slots while the other part of the bayonet connector includes guiding pins. Linearly pushing the spiral slots against the guiding pins imparts rotational movement to one part of the connector relative to the other part. Turning again to FIGS. 8A-8B and 9A-9B, the spiral design of bayonet slots 850, 860, 870 and 880 (in conjunction with the bayonet pins 960, 970, 980 and 990) enables rotating PEM 810, hence entire plunger head 800, about longitudinal axis 830 when PEM 810 is engaged with PRM 900 and linearly moved by plunger rod 700 relative to PRM 900. For example, when PEM 810 and PRM 190 are engaged, rotation of PEM 810 relative to PRM 190 is obtained by linearly pushing PEM 810 against PRM 900 when plunger rod 700 is to be released from PEM 810 (per configuration #2), or by linearly pushing PEM 810 away from (out of) PRM 900 to lock plunger rod 700 in PEM 810 (per configuration #3).

In configuration #2, plunger rod 700 is engageable with PEM 810 by using a snap-fitting connection (so, no rotation of any part relative to another part is required), and releasable from PEM 810 by rotating PEM 910 in a ‘release’ direction to release distal end 710 of plunger rod 700 from the ribs 820 of PEM 810. In configuration #3, PEM 810 is rotatable in a ‘lock’ direction to engage plunger rod 700 with PEM 810, in which every fin of plunger rod 700 is locked by a respective rib 820 of PEM 810, and in the ‘release’ direction to release plunger rod 700 from PEM 810.

In both configurations #2 and #3 rotating PEM 810 in the ‘release’ direction is implemented by axially retracting PEM 810 backwards by plunger rod 700, during which process bayonet slots 850, 860, 870 and 880 are respectively rotationally guided by bayonet pins 960, 970, 980 and 990 of PRM 900 to the ‘release’ angular position. In configuration #3, rotating PEM 810 in the ‘lock’ direction (i.e., in the direction opposite to the ‘release’ direction) is implemented by axially pushing PEM 810 forward by plunger rod 700, during which process bayonet slots 850, 860, 870 and 880 are rotationally guided by the bayonet pins of PRM 900 to the ‘lock’ angular position.

As described herein, PRM 900 is fixedly mounted to end point 126 of reservoir 120, which means that PRM 900 is stationary in reservoir 120. Referring to FIGS. 8A and 9A, after bayonet pins 960, 970, 980 and 990 respectively enter bayonet slots 850, 860, 870 and 880 via the respective entrance points, PEM 810 can be pushed linearly (axially) by plunger rod 700 in a first direction (i.e., to facilitate disengagement of plunger rod 700 from PEM 810, per the configuration #2) to thereby cause PEM 810 to rotate from the lock angular position in which each of the four ribs 820 locks one of fins 722, 724, 726 and 728, to the release (unlock) angular position in which none of fins 722, 724, 726 and 728 is locked by a rib and, therefore, plunger rod 700 can be disconnected from PEM 810.

When bayonet pins 960, 970, 980 and 990 respectively reside in bayonet slots 850, 860, 870 and 880, PEM 810 can be pushed longitudinally (axially) by plunger rod 700 in a second direction (i.e., to facilitate engagement of plunger rod 700 with PEM 810, per configuration #3) to cause PEM 810 to rotate from the release angular position, in which none of the fins of the plunger rod is locked by a rib 820, to the lock angular position in which all of fins 722, 724, 726 and 728 are respectively locked by ribs 820. Then, fins 722, 724, 726 and 728 can be released from ribs 820 by causing PEM 810 (by plunger rod 700, jointly with the bayonet pins) to rotate in the opposite direction (i.e., in the first direction), for example as described herein in connection with configuration #2.

In configuration #2, cylindrical member 840 of PEM 810 may be reversibly expandable (and/or ribs 820 reversibly compressible) to enable engagement between the plunger rod and PEM 810 by snap fitting the N ribs into segmented lock groove 740 of plunger rod 700 when plunger rod 700 is linearly moved (e.g., by motor 170) in PEM 800. In this configuration, plunger rod 700 is releasable from PEM 800 by rotating PEM 800 about its axis (830) from the ‘lock’ (‘snap-fit’) position to a ‘release’ position. In configuration #3, PEM 800 (including cylindrical member 840 and ribs 820) is made of a rigid (non-expandable) material, and locking the N fins (e.g., four fins, as shown in FIG. 7) by the N ribs (e.g., four ribs, as in FIG. 8A) is performed by rotating PEM 800 in a first direction (e.g., counterclockwise) about longitudinal axis 830, relative to entrance tip 720 of plunger rod 700, until fins 722, 724, 726 and 728 reach a ‘lock’ position in which each fin is locked by one of ribs 820. In this configuration, plunger rod 700 is releasable from PEM 800 by rotating PEM 800 about its axis (830) in the opposite direction (e.g., clockwise), from the ‘lock’ position to a ‘release’ position.

The fins, ribs, and the recesses that are formed by the ribs are configured such that plunger rod 700 is transitionable from a lock angular position, in which the fins are respectively aligned with, and locked by, the ribs to enable backward movement of PEM 800 (hence the plunger head) in unison with the retraction movement of plunger rod 700, to an unlock (release) angular position in which the fins are respectively aligned with the recesses to enable releasing entrance tip 720 of plunger rod 700 from PEM 800, hence from the plunger head.

Referring to FIG. 7 and FIG. 10A, distal end 710 of plunger rod 700 also includes a pushing flange 760. Flange (ledge) 950 of PRM 900 (FIGS. 9A-9B) has an internal diameter D8, flange 760 of plunger rod 700 has an outer diameter D9, and PEM 810 has an internal diameter D10. The values of D8, D9 and D10 are selected such that D10<D9<D8, so that when plunger rod 700 is moved axially (e.g., by motor 170) in direction 1010 (at this stage without moving PEM 810), entrance tip 720 of plunger rod 700 linearly (axially) moves through inner flange (ledge) 950 of PRM 900 and, then, through cylindrical member 840 of PEM 810 until flange 760 contacts (abuts) cylindrical member 840 of PEM 810 at 1020. From this point on, linearly moving plunger rod 700 further in direction 1010 starts moving PEM 810 in the same direction (in direction 1010). The bayonet slots and pins are designed such that the linear displacement of PEM 810 (hence of plunger head 800 as a whole) in direction 1010 is converted into rotational movement (1030) of PEM 810 about its axis (axis 830). As PEM 810 is pushed further linearly, it continues its rotational movement about axis 830 until the bayonet slots respectively depart (moved away) from the stationary bayonet pins.

Diameter D9 of flange 760, by being larger than inner diameter D10 of cylindrical member 840, enables flange 760 to push PEM 810 (which is part of the plunger head) concomitantly with plunger rod 700 in the forward (emptying) direction 1010 as plunger rod 700 is moved forward in reservoir 1020 by motor 170. In both configuration #2 and configuration #3 when plunger rod 700 is locked in/by PEM 810, PEM 810 is tightly sandwiched between pushing flange 760 of plunger rod 700 and the pulling ledges of fins 720, and bidirectionally moveable in reservoir 2020 in unison with plunger rod 700: in direction 1010 to empty reservoir 120, and in the opposite direction to fill up reservoir 120.

FIG. 10B shows an initial state where fins 722, 724, 726 and 728 of plunger rod 700 are unlocked by ribs 820 so that plunger rod 700 can be retracted from PEM 810 by moving plunger rod linearly. For example, fin 722 is free to bidirectionally move through recess 1040 that is formed by ribs 820/1 and 820/2. Similarly, fin 728 is free to bidirectionally move through recess 1050 that is formed by ribs 820/4 and 820/1. FIG. 10C shows the final angular state in which the bayonet slots have respectively departed (moved away) from the stationary bayonet pins and PEM 810 has been rotated relative to fins 722, 724, 726 and 728 of plunger rod 700 to an angular position in which fins 722, 724, 726 and 728 are respectively locked by ribs 820 of PEM 800.

FIG. 10D schematically illustrates an example initial angular position (γ) of an example PEM 1060 relative to fins of an example plunger rod. By way of example, PEM 1060 includes four ribs (1062, 1064, 1066, and 1068), so the plunger rod includes four fins 1070. The initial angular position (angle γ) of PEM 1060 relative to the plunger rod (hence to fins 1070) is referred to herein as ‘unlock angular position’, or ‘release angular position’, because neither of fins 1070 is locked by a rib of the PEM, so, in this angular position of PEM 1060 relative to the plunger rod fins 1070 are retractable from PEM 1060. While the value of angle δ between each two adjacent ribs is constant, the value of angle γ between a rib and a fin adjacent to the rib changes according to the linear (axial) distance travelled by the plunger rod (hence by the PEM). The value of & depends on the number of ribs (r) of the PEM, and may be calculated as follows: δ=360°/r. In the example shown in FIG. 10D there are four ribs (r=4), so δ=360°/4=90°. The value of γ may be calculated as follows: γ=δ/2. Continuing the example above, γ=δ/2=90°/2=45°.

FIG. 10E schematically illustrates PEM 1060 after plunger rod 700, by pushing PEM 1060 linearly, caused PEM 1060 to rotate counterclockwise (CCW) by angle γ=45°, from the unlock/release angular position shown in FIG. 10D (which corresponds to the unlock/release angular position shown in FIG. 10B) to the lock angular position (which corresponds to the lock angular position shown in FIG. 10C) where each of fins 1070 is locked by a corresponding rib. For example, fin 1072 is locked by rib 1068. (The four ‘black and white’ dotted patterns shown in FIG. 10E, for example black and white dotted pattern 1080, denote distal areas of the fins that respectively overlap, i.e., contact, distal areas of the ribs. In FIG. 10D, the fins and the ribs do not overlap.) FIGS. 10B, 10C and 10C show a fin structure that includes four fins, and a consequential rib structure that includes four ribs. However, a fin structure of a plunger rod may include less than four fins (for example three fins) or more than four fins (for example five fins), with the ribs structure having an identical number of ribs.

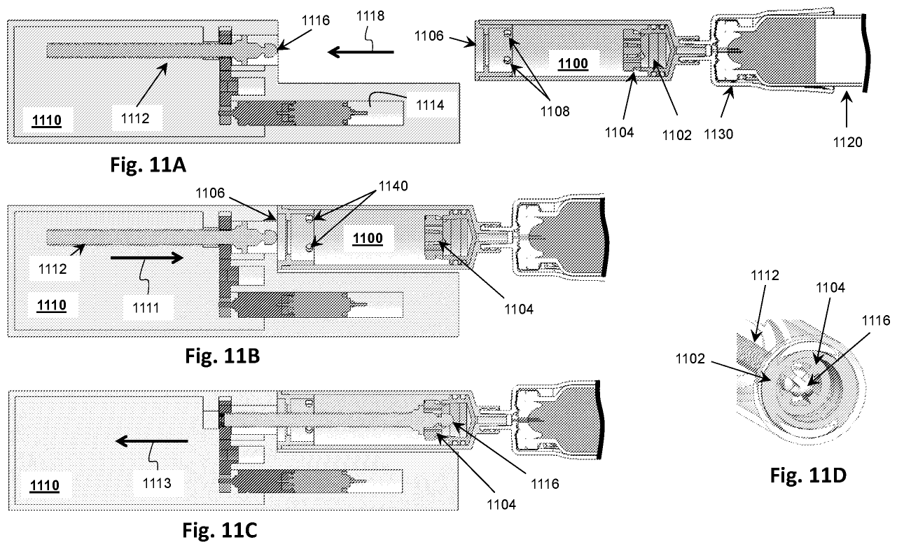

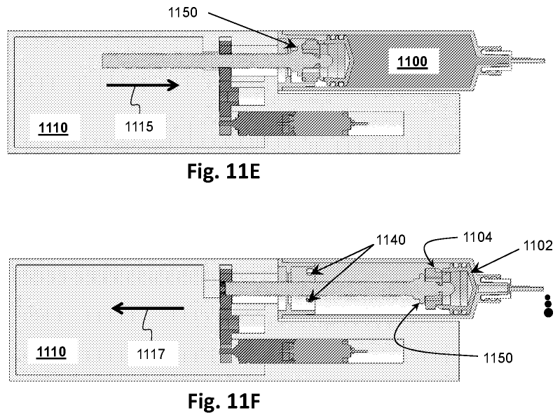

FIGS. 11A-11I show stages in operating a pump device in according with configuration #2. FIG. 11A shows a disposable part (a reservoir) 1100 of a pump device before it is coupled to a reusable part 1110 of the pump device. Reservoir 1100 includes a plunger head 1102 that is bidirectionally moveable in reservoir 1100. Plunger head 1102 includes a plunger engagement member (PEM) 1104 that is structured as, or includes, a snap-fit connection (locking) mechanism.

Disposable part (reservoir) 1100 also includes a plunger release member (PRM) 1106 that is stationary in disposable part 1100, i.e., PRM 1106 is fixedly mounted in the proximal end of disposable part 1100 and is unable to move with respect to disposable part 1100. Reusable part 1110 of the pump device includes, among other things, plunger rod 1112 and an electric motor 1114 for linearly moving plunger rod 1112 bidirectionally. Plunger rod 1112 includes a distal end 1116 that, like distal end 710 of plunger rod 700, includes a pushing flange and a plurality of fins. PRM 1106 includes bayonet pins. (Two bayonet pins of PRM 1106 are shown at 1108.) Disposable part 1100 is coupled to a medical vial 1120 via vial adapter 1130. To start operating the pump device, disposable part 1100 is moved (1118) towards reusable part 1110 to couple them, for example magnetically.

Initially (e.g., before the disposable part and the reusable part are coupled), plunger rod 1112 is stowed in (fully retracted into) reusable part 1110. When disposable part 1100 is coupled to reusable part 1110, as shown in FIG. 11B, electric motor 1114 is controllably operated to move (extend) plunger rod 1112 forward, in direction 1111, to engage plunger rod 1112 with PEM 1104 (hence with plunger head 1102). Engaging plunger rod 1112 with PEM 1104 is implemented by snap-fitting ribs of PEM 1104 to the fins of distal end 1116 of plunger rod 1112, so that the ribs of PEM 1104 respectively lock the fins of plunger rod 1112.

Referring to configuration #2, the PEM (e.g., PEM 1104) may initially be positioned in the distal end of the reservoir, as shown, for example, in FIGS. 11A-1C. However, the PEM may initially be in the reservoir at any intermediate location between the PRM (e.g., PRM 1106) and the distal end of the reservoir. If the PEM is initially positioned at an intermediate location between the PRM and the distal end of the reservoir, the plunger rod may be required to push the PEM, hence the plunger head, to the distal end of the reservoir to ensure that the distal end (e.g., distal end 1116) of the plunger rod is properly snap fitted by the PEM.

FIG. 11C shows distal end 1116 of plunger rod 1112 fully extended and locked by PEM 1104 by being snap fitted by PEM 1104. (FIG. 11D shows the fins of plunger rod 1112 in an angular lock position in which the fins of the plunger rod are locked by the ribs of PEM 1104.) At this stage, electric motor 1114 is controllably operated to retract plunger rod 1112 backwards, in direction 1113. Since the fins of plunger rod 1112 are locked by the ribs of PEM 1104, retraction of plunger rod 1112 causes plunger head 1102 to move concomitantly with plunger rod 1112, which produces a suction force in reservoir 1100 that fills reservoir 1100 with medicament. (FIG. 11E shows reservoir 1100 filled with medicament.)

Referring to FIG. 11E, PEM 1104 is retracted (withdrawn by plunger rod 1112) in reservoir 1100 from the distal end of reservoir 1100 only up to a point where bayonet pins 1140 (FIG. 11B) of PRM 1106 respectively abut the entrance points of the slots in PEM 1104. Retraction of PEM 1104 further beyond the abutment points, for example to the stow position of PEM 1104 in PRM 1106, would cause bayonet pins 1140 of PRM 1106, jointly with the bayonet slots in PEM 1104, to rotate PEM 1104 about its axis from the lock angular position shown at FIG. 11D to the unlock (release) angular position shown in FIG. 11H, which is not desired at this stage. At this stage, electric motor 1114 (FIG. 11A) may be controllably operated to linearly (axially) move (extend) plunger rod 1112 forward, in direction 1115, to empty reservoir 1100, for example by delivering the medicament from reservoir 1100 to an infusion site of a patient. When plunger rod 1112 is linearly moved forward (for example by electric motor 1114), in direction 1115, flange 1150 of plunger rod 1112 pushes PEM 1104 in the same direction to deliver the medicament in reservoir 1100 to a person in need.

FIG. 11F shows reservoir 1100 in its empty state and with plunger rod 1112 fully extended. At this stage, electric motor 1114 is controllably operated to linearly retract plunger rod 1112 backwards, in direction 1117, to release plunger rod 1112 from PEM 1104 (hence from plunger head 1102). To release plunger rod 1112 from PEM 1104 plunger rod 1112 is fully retracted in reservoir 1100 (as opposed to the partial retraction of the plunger rod in FIG. 11E) to operationally engage PEM 1104 with PRM 1108 in order to cause bayonet pins 1140 and the bayonet slots in PEM 1104 to rotate PEM 1104 from the lock angular position to the unlock (release) angular position while PEM 1104 is moved by plunger rod 1112 linearly, in direction 1117.

FIG. 11G shows plunger rod 1112 fully retracted in reservoir 1100, in which position PEM 1104 is completely stowed in PRM 1106. FIG. 11H shows the ribs of PEM 1104 in the unlock angular position in which plunger rod 1112 (FIG. 11D) can be released from PEM 1104. At this stage, plunger rod 1112 can be fully retracted by the electric motor back into reusable part 1110, and disposable part 1100 decoupled (disconnected) from reusable part 1110. FIG. 11I shows plunger rod 1112 fully retracted (completely stowed) in reusable part 1110, PEM 1104 completely stowed in PRM 1106, and reusable part 1110 decoupled from disposable part 1100. So, stowing PEM 1104 in PRM 1106 by motor 1114 automatically ejects plunger rod 1112 from PEM 1104. So, PRM 1106, by functioning both as an ejection means and as a stowing means, ejects plunger rod 1112 from PEM 1104 when plunger rod 1112 pulls PEM 1104 into the stow position in PRM 1106. (The ejection of plunger rod 1112 from PEM 1104 is performed by longitudinally rotating PEM 1104 relative to plunger rod 1112.)

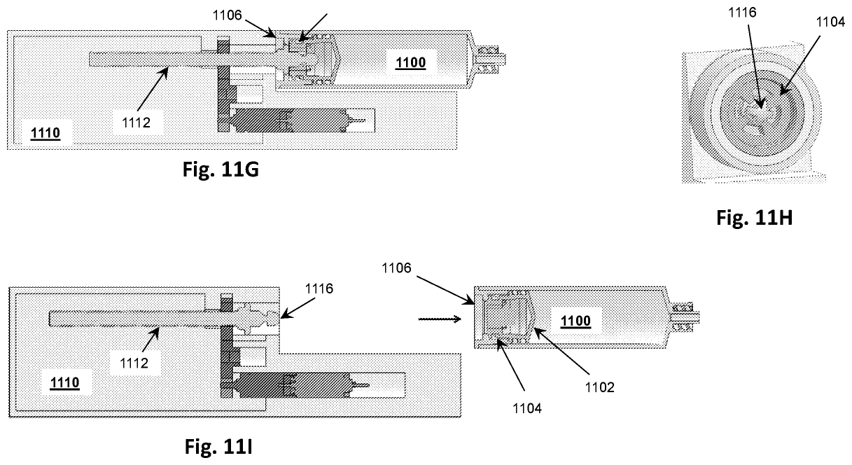

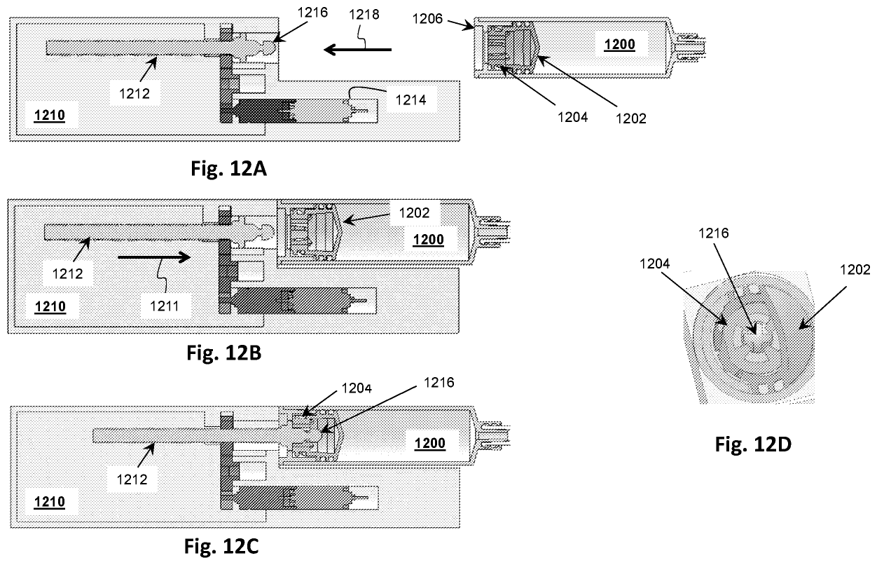

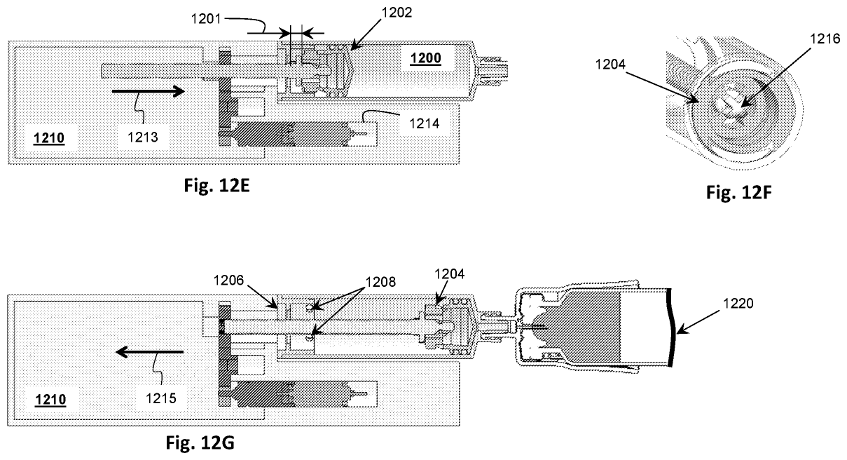

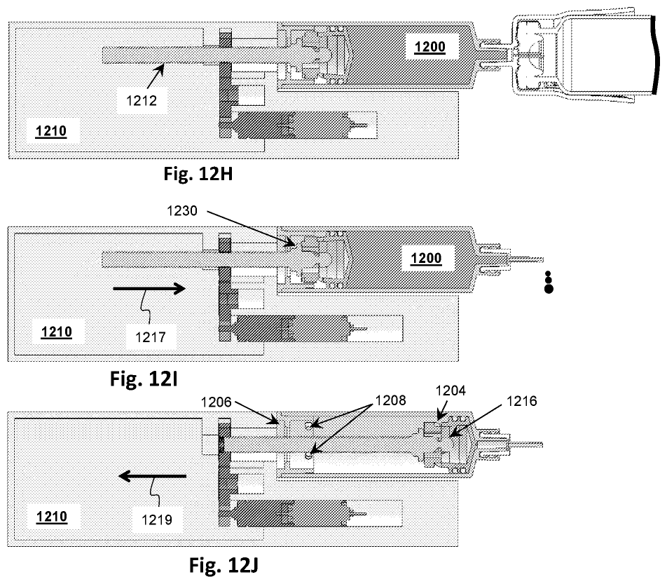

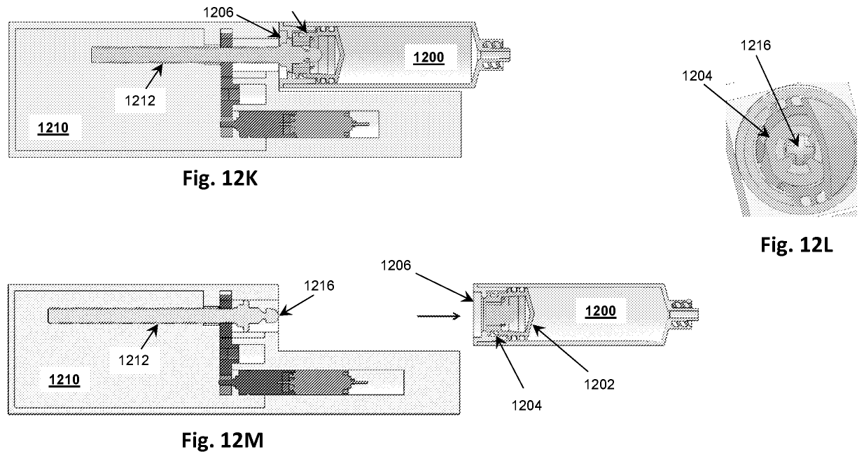

FIGS. 12A-12M show stages in operating a pump device in according with configuration #3. FIG. 12A shows a disposable part (a reservoir) 1200 of a pump device before it is coupled to a reusable part 1210 of the pump device. Reservoir 1200 includes a plunger head 1202 that is bidirectionally moveable in reservoir 1200. Plunger head 1202 includes a plunger engagement member (PEM) 1204 that is structured as, or includes, a bayonet connection mechanism.

Disposable part (reservoir) 1200 also includes a plunger release member (PRM) 1206 that is stationary in disposable part 1200, i.e., PRM 1206 is fixedly mounted in the proximal end of disposable part 1200 and is unable to move with respect to disposable part 1200. Reusable part 1210 of the pump device includes, among other things, plunger rod 1212 and an electric motor 1214 for linearly moving plunger rod 1212 bidirectionally. Plunger rod 1212 includes a distal end 1216 that, like distal end 710 of plunger rod 700, includes a pushing flange and a plurality of fins. PRM 1206 includes bayonet pins. (The bayonet pins of PRM 1206 are shown in FIGS. 12G and 12J, at 1208.) To start operating the pump device, disposable part 1200 is moved in direction 1218, towards reusable part 1210 to couple them, for example magnetically. Initially (e.g., before disposable part 1200 and reusable part 1210 are coupled), plunger rod 1212 is stowed in (fully retracted into) reusable part 1210, and PEM 1204 is completely stowed in PRM 1206.

When disposable part 1200 is coupled to reusable part 1210, as shown in FIG. 12B, electric motor 1214 is controllably operated to move (extend) plunger rod 1212 forward, in direction 1211, to engage distal end 1216 of plunger rod 1212 with PEM 1204 (hence with plunger head 1202). Engaging distal end 1216 of plunger rod 1212 with PEM 1204 is implemented by using a bayonet mechanism that enables the ribs in PEM 1204 to rotate relative to the fins in distal end 1216 of plunger rod 1212, from an unlock angular position to a lock angular position, so that in the angular lock position the ribs of PEM 1204 respectively lock the fins of plunger rod 1212.

In configuration #3, PEM 1204 is initially positioned (stowed) in the proximal end of reservoir 1200, with the bayonet pins of PRM 1206 respectively already resting in (occupying) the helical bayonet slots of PEM 1204, as demonstrated, for example, by FIG. 12A-2C. A linear movement of PEM 1204 away from the initial (stow) position would cause the bayonet mechanism, namely, the slots in PEM 1204 in conjunction with pins 1208 (FIGS. 12G and 12J) in PRM 1206 to rotate PEM 1204 from the unlock angular position to the lock angular position.

FIG. 12C shows distal end 1216 of plunger rod 1212 residing in PEM 1204 in a pre-lock state, namely, before PEM 1204 is pushed against bayonet pins 1208 to rotate PEM 1204 to the lock angular position. FIG. 12D shows distal end 1216 of plunger rod 1212 (hence the fins of plunger rod 1212) still unlocked by the ribs of PEM 1204.

FIG. 12E shows PEM 1204 linearly moved (by electric motor 1214) away a distance 1201 from the stow position in PRM 1206 and consequentially rotated from the unlock angular position to the lock angular position. In the lock angular position, the fins of plunger rod 1212 are respectively locked by the ribs of PEM 1204, as shown in FIG. 12F. At this stage, electric motor 1214 is controllably operated to move plunger rod 1212 forward further, in direction 1213, in preparation for filling reservoir 1200 with medicament by sucking the medicament from a medicament vial.

FIG. 12G shows PEM 1204 at the distal end of reservoir 1200, and a vial 1220 coupled to reservoir 1200 via a vial adaptor. Since at this stage the fins of plunger rod 1212 are locked by the ribs of PEM 1204, retraction of plunger rod 1212 in direction 1215 causes plunger head 1202 to move in unison with plunger rod 1212 in direction 1215. Movement of plunger head 1202 in direction 1215 produces a suction force in reservoir 1200 that fills reservoir 1200 with medicament. (FIG. 12H shows reservoir 1200 filled with medicament.)

Referring to FIG. 12H, PEM 1204 is retracted (withdrawn by plunger rod 1212) in reservoir 1200 from the distal end of reservoir 1200 only up to a point where the bayonet pins of PRM 1206 respectively abut the entrance points of the slots in PEM 1204. (At this point reservoir 1200 is full of medicament.) Retraction of PEM 1204 further, beyond the abutment points, for example to the stow position of PEM 1204 in PRM 1206, would cause bayonet pins 1208 in PRM 1206, jointly with the slots in PEM 1204, to rotate PEM 1204 about its axis from the lock angular position shown at FIG. 12F to the unlock (release) angular position shown in FIG. 12L, which is not desired at this stage. At this stage, electric motor 1214 may be controllably operated to linearly (axially) move (extend) plunger rod 1212 forward, in direction 1217, as shown in FIG. 12I, to empty reservoir 1200, for example by delivering the medicament from reservoir 1200 to an infusion site of a patient. When plunger rod 1212 is linearly moved forward (for example by electric motor 1214) in direction 1217, flange 1230 of plunger rod 1212 pushes PEM 1204 in the same direction to deliver the medicament in reservoir 1200 to a person in need.

FIG. 12J shows reservoir 1200 in it is empty state and with plunger rod 1212 fully extended. At this stage, the electric motor is controllably operated to linearly retract plunger rod 1212 backwards, in direction 1219, to release distal end 1216 of plunger rod 1212 from PEM 1204 (hence from plunger head 1202). To release distal end 1216 of plunger rod 1212 from PEM 1204 plunger rod 1212 is fully retracted in reservoir 1200 (as opposed to partial retraction of plunger rod 1212 in FIG. 12H) in order to (re-)engage PEM 1204 with PRM 1206 in order to cause bayonet pins 1208 and the bayonet slots in PEM 1204 to rotate PEM 1204 from the lock angular position to the unlock (release) angular position while PEM 1204 is moved by plunger rod 1212 linearly in direction 1219.

FIG. 12K shows plunger rod 1212 fully retracted in reservoir 1200, and FIG. 12L shows plunger rod 1212 released from the ribs of PEM 1204 after rotating PEM 1204 (relative to the plunger rod) to the unlock angular position in which plunger rod 1212 can be released from PEM 1204. At this stage, plunger rod 1212 can be fully retracted by the electric motor back and stowed in reusable part 1210, and disposable part 1200 can be decoupled (disconnected) from reusable part 1210, as shown in FIG. 12M. So, stowing PEM 1204 in PRM 1206 by motor 1214 automatically ejects plunger rod 1212 from PEM 1204 in a similar way as described in connection with FIG. 11G.

FIG. 12K shows plunger rod 2112 fully retracted in reservoir 1200, in which position PEM 1204 is completely stowed in PRM 1206. FIG. 12L shows the four ribs of PEM 1204 in the unlock angular position in which plunger rod 1212 can be released from PEM 1204. At this stage, plunger rod 1212 can be fully retracted back by the electric motor and stowed in reusable part 1210, and disposable part 1200 can be decoupled (disconnected) from reusable part 1210. FIG. 12M shows plunger rod 1212 fully retracted (completely stowed) in reusable part 1210, PEM 1204 completely stowed in PRM 1206, and disposable part (reservoir) 1200 decoupled from reusable part 1210.

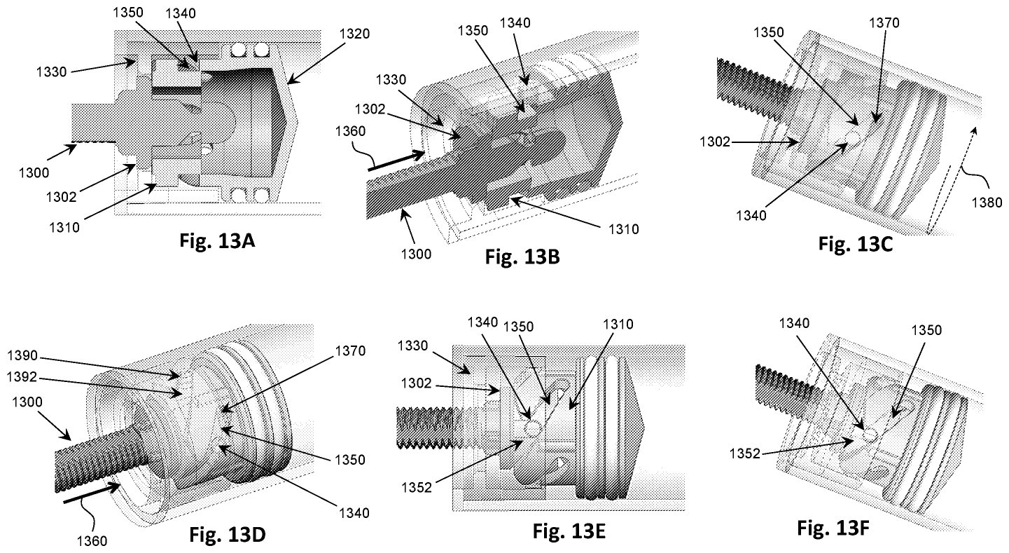

FIGS. 13A-13F demonstrate using a bayonet feature for locking a plunger rod 1300 in a plunger engagement member (PEM) 1310 of a plunger head 1320 according to configuration #3. FIG. 13A is a cross-sectional view showing PEM 1310 in a stow position in plunger release member (PRM) 1330. As described herein in connection with configurations #2 and #3, when the PEM is in the stow position in the PRM, the PEM is in the unlock (release) angular position, which means that the plunger rod is not yet locked by the PEM. In addition, when the PEM is in the stow position in the PRM, each bayonet pin of the PRM is located at the end point of the respective bayonet slot in the PEM.

Referring to FIG. 13A, bayonet pin 1340 of PRM 1330 is located at the end point of bayonet slot 1350 in PEM 1310. (The end point of bayonet slot 1350 is more clearly shown in FIG. 13C, at 1370.) At this stage the bayonet pins (e.g., pin 1340) are respectively the farthest from the entrance points of the bayonet slots. FIG. 13B shows a cross-sectional view of a three-dimensional assembly including plunger rod 1300, PEM 1310 and PRM 1330.

Linearly moving plunger rod 1300 in direction 1360 (FIG. 13B), for example by an electric motor, causes flange 1302 of plunger rod 1300 to push PEM 1310 in the same direction (1360) and, consequently, to rotate PEM 1310 in the counterclockwise (CCW) direction 1380 (FIG. 13C). When PEM 1310 starts rotating in the CCW direction, the end points of the bayonet slots respectively depart from the bayonet pins, and the distance between each slot's end point and the respective bayonet pin grows as PEM 1310 continues to rotate in the CCW direction (1380). FIG. 13C shows PEM 1310 in an intermediate state where the bayonet pins are positioned about halfway up the bayonet slots. By way of example, FIG. 13C shows bayonet pin 1340 in an intermediate state where bayonet pin 1340 is about halfway up bayonet slot 1350. FIG. 13D shows the assembly of FIG. 13C from a different perspective and an additional bayonet pin (pin 1390) that, like bayonet pin 1340, is about halfway up the corresponding bayonet slot (slot 1392).

As plunger rod 1300 continues to be moved linearly in direction 1360, its flange 1302 continues to push PEM 1310 in the same direction. As a result of the linear (axial) movement of PEM 1310 the pins of PRM 1330 continue to rotate PEM 1310 in the CCW direction until PEM 1310 reaches its lock angular position. When PEM 1310 reaches its lock angular position, the fins of plunger rod 1300 are respectively locked by the ribs of PEM 1310, as demonstrated, for example, in FIG. 12F. (FIG. 12F shows PEM 1204 in its lock angular position in which the fins of plunger rod 1212 are respectively locked by the ribs of PEM 1204.)

FIG. 13E shows PEM 1310 (hence plunger head 1320 as a whole) in its lock angular position in which the bayonet pins of PRM 1330 are respectively positioned at the entrance point of the bayonet slots in PEM 1310, ready to be left by the bayonet slots. By way of example, bayonet pin 1340 is positioned at entrance point 1352 of bayonet slot 1350. When PEM 1310 reaches its lock angular position, further movement of PEM 1310 in direction 1360 causes PEM 1310 to move linearly to the distal end of the reservoir without being rotated any further in the CCW direction, or in the clockwise (CW) direction. (When PEM 1310 is moved between the proximal end and the distal end of the reservoir, PEM 1310 does not rotate to either direction (CCW or CW) but, rather, maintains its lock angular position.)

Regarding configurations #2 and #3, the number of bayonet pins and bayonet slots is not limited to any specific number (for example to two, three or four), and the number of bayonet pins is identical to the number of bayonet slots so that with each particular bayonet slot is associated one bayonet pin, and vice versa.

According to some embodiments the disposable reservoir contains levodopa or carbidopa, or a combination of levodopa and carbidopa.

The benefits of a disposable part (reservoir) that includes the PEM and PRM of the present invention comparing to conventional disposable parts (reservoirs) are at least the following:
1. Reduced cost of goods (COGS) related to disposable part (reservoir) of a pump device;
2. Fewer parts that are embedded, or included, in the disposable part (reservoir);
3. Improved product sustainability;
4. Higher precision propulsion system due to the leadscrew (plunger rod) being part of the reusable part of the pump device, which enables operating it with higher precision and smaller, or tightened, tolerances;
5. Ability to design a rounded syringe, as opposed to oval syringe or syringes with other shapes;
6. Simplified assembly process of the disposable part (reservoir);
7. Supporting pre-filled reservoir concept;
8. Higher leadscrew positioning and position monitoring accuracy;
9. More efficient occlusion control due to enhanced control of the position of the lead screw (plunger rod), and
10. Improved medicament transfer system concept.

The articles “a” and “an” are used herein to refer to one or to more than one (e.g., to at least one) of the grammatical object of the article, depending on the context. By way of example, depending on the context, “an element” can mean one element or more than one element. The term “including” is used herein to mean, and is used interchangeably with, the phrase “including but not limited to”. The terms “or” and “and” are used herein to mean, and are used interchangeably with, the term “and/or,” unless context clearly indicates otherwise. The term “such as” is used herein to mean, and is used interchangeably, with the phrase “such as but not limited to”.

Having thus described exemplary embodiments of the invention, it will be apparent to those skilled in the art that modifications of the disclosed embodiments will be within the scope of the invention. Alternative embodiments may, accordingly, include functionally equivalent objects/articles. For example, the PEM and/or PRM and/or the plunger rod may have a different design (e.g., different shape, size and/or material) than the ones shown in the drawings. Features of certain embodiments may be used with other embodiments shown herein. The present disclosure is described in connection with pump devices that include a disposable reservoir and a reusable part. However, the present disclosure may be relevant to (e.g., it may be implemented by, used with or for) other types of ‘two-part’ devices, pumps, syringes, therapeutic drug dispensing devices, and the like. Hence the scope of the claims that follow is not limited by the disclosure herein.

1. A pump device for delivering medicament, comprising:
a disposable reservoir comprising:
a plunger head bidirectionally moveable in the disposable reservoir between a proximal end of the disposable reservoir and a distal end of the disposable reservoir; and
a reusable part comprising a plunger rod, wherein the plunger rod and the plunger head are configured such that the plunger rod is releasably lockable in the plunger head when the disposable reservoir and the reusable part are coupled to one another.

2. The pump device of claim 1, wherein the plunger head comprises a concentric plunger engagement member (PEM), the PEM bidirectionally moveable in unison (en masse) with the plunger head between the proximal end of the disposable reservoir and the distal end of the disposable reservoir, and wherein the disposable reservoir further comprises a bored plunger release member (PRM), said PRM concentrically and fixedly mounted to the proximal end of the disposable reservoir, and wherein the plunger rod comprises a distal end and bidirectionally moveable through the bored PRM, and wherein when the disposable reservoir and the reusable part are coupled to one another the distal end of the plunger rod is lockable in the PEM and releasable from the PEM by the PRM.

3. The pump device of claim 1, wherein the plunger rod is lockable in and releasable from the PEM by using a snap-fit connection means, or a bayonet connection means, or a combination of snap-fit connection means and bayonet connection means.

4. The pump device of claim 3, wherein the snap-fit connection means comprises an annular snap-fit feature, or a cantilever snap-fit connection feature, or a combination of them.

5. The pump device of claim 3, wherein the snap-fit connection means comprises the distal end of the plunger rod, the PEM and the PRM.

6. The pump device of claim 5, wherein the distal end of the plunger rod comprises an entrance tip and an annular lock groove circumferentially formed in the distal end between the entrance tip and the rest of the plunger rod, and wherein the PEM comprises a ring-shaped base and flexible arms circularly extending from the ring-shaped base and bending inwardly towards a central axis of the PEM to form a flexible cap-like structure with an opening, said flexible cap-like structure is snap-fittable into the annular lock groove.

7. The pump device of claim 6, wherein the PRM comprises an external (outer) cylindrical body and a concentric inner (internal) hollow cylindrical body through which the plunger rod is movable, wherein the external cylindrical body and the concentric inner hollow cylindrical body define, therebetween, an open annular channel, and wherein the inner hollow cylindrical body is configured to deflect the flexible arms of the PEM radially outward to release (‘snap-out’) the plunger rod from the PEM when the PEM is retracted by the plunger rod into a stow position in the open annular channel.

8. The pump device of claim 6, wherein the entrance tip is selected from the group consisting of: hemispherical tip, conical tip, truncated trapezoid.

9. The pump device of claim 6, wherein each flexible arms comprises a proximal section that extends from the ring-shaped base at an acute angle α relative to a plane of the ring-shaped base (where α<90°), and a distal section that contiguously extends from the proximal section at an acute angle β relative to the ring-shaped base (where β<α).

10. The pump device of claim 9, wherein the distal sections of the flexible arms are configured to snap-fit into the annular lock groove of the plunger rod.

11. The pump device of claim 10, wherein the annular lock groove comprises an annular pulling ledge for pulling the PEM in unison with the plunger rod in a backward direction by pulling the distal sections of the flexible arms when the plunger rod is retracted in the disposable reservoir.

12. The pump device of claim 6, wherein the annular lock groove further comprises a conical surface to enable movement of the plunger rod through the opening in the flexible cap-like structure in a forward direction until the entrance tip of the plunger rod contacts an inner push surface of the plunger head.

13. The pump device of claim 6, wherein as the entrance tip of the plunger rod is moved forward through the opening in the flexible cap-like structure the flexible arms are deflected radially outward by the entrance tip of the plunger rod and, as the entrance tip is moved passed the opening in the flexible cap-like structure, the flexible cap-like structure is snap-fitted into the annular lock groove in a way that locks the plunger rod in the plunger engagement member (PEM).

14. The pump device of claim 6, wherein the entrance tip of the plunger rod comprises a curved entrance side to deflect the flexible arms radially outward.

15. The pump device of claim 6 wherein the number of flexible arms is 2n (where n=1, 2, 3, . . . , etc.), and wherein the flexible arms are equally sized and shaped, and are angularly equidistant from one another.

16. The pump device of claim 6, wherein the number of flexible arms is selected from the group consisting of: four (4) flexible arms, six (6) flexible arms, and eight (8) flexible arms.

17. The pump device of claim 3, wherein the snap-fit connection is a non-reversible snap-fit connection.

18. The pump device of claim 2, wherein the distal end of the plunger rod comprises:
an entrance tip comprising a number N of fins angularly distributed around and radially extending outward from a longitudinal axis of the plunger rod, and a segmented (discrete, discontinued) lock groove circumferentially formed in the distal end between the N fins and the rest of the plunger rod, and
wherein the plunger engagement member (PEM) comprises:
a bored cylindrical member having an inner wall;
a number N of ribs radially extending inward from the inner wall toward a longitudinal axis of the bored cylindrical member and configured to respectively lock the N fins; and
a number S of bayonet slots circumferentially formed in the bored cylindrical member.

19. The pump device of claim 18, wherein each of the S bayonet slots is designed as helix that encircles the longitudinal axis of the bored cylindrical member at a constant distance from the longitudinal axis, said bayonet slots enabling the PEM to rotate about the longitudinal axis of the bored cylindrical member by a rotational angle suitable to lock the N fins by the N ribs and to unlock the N fins from the N ribs.

20. The pump device of claim 18, wherein the PRM comprises a hollow cylindrical body through which the plunger rod is movable, and a number S of bayonet pins radially extending inward from an inner wall of the hollow cylindrical body, wherein the S bayonet pins are respectively engageable with the S bayonet slots of the PEM to rotate the PEM relative to the PRM

21. The pump device of claim 20, wherein the PEM is rotatable relative to the PRM between:
a lock angular position, in which the N fins are respectively aligned with, and locked by, the N ribs to enable retraction of the plunger head by the plunger rod, and
a release angular position, in which the N fins are respectively misaligned with the N ribs to enable releasing the plunger rod from the plunger head when the PEM is linearly retracted by the plunger rod into a stow position in the PRM,
wherein each of the S bayonet slots is designed to enable the rotation of the PEM from the lock angular position to the release angular position when the PEM is linearly moved into the stow position in the PRM, and from the release angular position to the lock angular position when the PEM is linearly moved away from the stow position in the PRM.

22. The pump device of claim 21, wherein rotating the PEM from the lock angular position to the release angular position comprises rotating the PEM clockwise (or counterclockwise) by a release angle (γ), and wherein rotating the PEM from the release angular position to the lock angular position comprises rotating the PEM counterclockwise (or clockwise) by a lock angle (γ), wherein the value of the release/lock angle (γ) is γ=180°/N.

23. The pump device of claim 22, wherein the value of N is selected from the group consisting of: N=2, N=3, N=4, N=5, and N=6.

24. The pump device of claim 21, wherein the distal end of the plunger rod further comprises a pushing flange, the pushing flange having a diameter larger than an inner diameter of the bored cylindrical member to enable pushing the PEM in unison with the plunger rod in the forward direction as the plunger rod is moved forward in the disposable reservoir.

25. The pump device of claim 24, wherein the N fins comprise N pull ledges and wherein in the lock angular position the PEM is sandwiched between the pushing flange and the N pull ledges and moveable in unison with the plunger rod in a backward direction as the plunger rod is retracted in the disposable reservoir.

26. The pump device of claim 18, wherein the entrance tip is shaped as a segmented hemispherical tip, segmented conical tip, or as a cruciform Phillips screwdriver head.

27. The pump device of claim 21, wherein the PEM is initially positioned at the distal end of the disposable reservoir and the plunger rod is lockable by the PEM in the lock angular position by moving the plunger rod forward in the disposable reservoir, through the PRM, until the N fins of the plunger rod are respectively snap-fitted by the N ribs of the PEM, and wherein the plunger rod is releasable from the PEM by retracting the PEM to the stow position in the PRM to cause a twisting motion of the PEM, relative to the PRM, from the lock angular position to the release angular position.

28. The pump device of claim 27, wherein the bored cylindrical member of the PEM is reversibly expandable by the N fins of the plunger rod to facilitate the snap-fitting of the N ribs of the PEM into the segmented lock groove in the distal end of the plunger rod.

29. The pump device of claim 27, wherein the N ribs of the PEM are reversibly compressible by the N fins to facilitate the snap-fitting of the N ribs of the PEM into the segmented lock groove in the distal end of the plunger rod.

30. The pump device of claim 21, wherein the PEM is initially in the stow position in the PRM in the release angular position, and wherein the plunger rod is lockable by the PEM by moving the plunger rod forward through the PRM such that the plunger rod linearly moves the PEM away from the stow position in the PRM while simultaneously causing the PEM to rotate from the release angular position to the lock angular position, and wherein the plunger rod is releasable from the PEM by retracting the PEM back to the stow position in the PRM to rotate the PEM from the lock angular position to the release angular position.

31. A disposable reservoir releasably couplable to a reusable part of a drug delivery device, the disposable reservoir comprising:
a plunger head, said plunger head comprising a plunger engagement member (PEM) and bidirectionally moveable in unison with the PEM between a proximal end of the disposable reservoir and a distal end of the disposable reservoir, said PEM configured to engage a plunger rod of the reusable part of the drug delivery device; and
a plunger release member (PRM) fixedly attached to the proximal end of the disposable reservoir, said PRM configured to release the plunger rod from the PEM when the PEM is moved by the plunger rod to a stow position in the PRM.

32. The disposable reservoir of claim 31, wherein the plunger rod is lockable by or in the PEM by moving the plunger rod forward in the disposable reservoir, through the PRM, until the plunger rod is snap fitted by flexible arms of the PEM, and wherein the plunger rod is releasable from the PEM by retracting the PEM to the stow position where the PRM deflects the flexible arms of the PEM to release the plunger rod from the PEM.

33. The disposable reservoir of claim 31, wherein the PEM is initially positioned at the distal end of the disposable reservoir and the plunger rod is initially lockable by the PEM in a lock angular position by moving the plunger rod forward in the disposable reservoir, through the PRM, until the plunger rod is snap fitted by the PEM, and wherein the plunger rod is releasable from the PEM by moving the PEM to the stow position in the PRM to cause a twisting motion of the PEM, relative to the PRM, from the lock angular position to a release angular position.

34. The disposable reservoir of claim 31, wherein the PEM is initially stowed in the PRM in a release angular position, and wherein the plunger rod is lockable by the PEM by moving the plunger rod forward through the PRM such that the plunger rod linearly moves the PEM away from the stow position in the PRM while simultaneously causing the PEM to rotate from the release angular position to a lock angular position, and wherein the plunger rod is releasable from the PEM by moving the PEM back to the stow position in the PRM to rotate the PEM from the lock angular position back to the release angular position.

35. The device of claim 1 or claim 31, wherein the disposable reservoir contains levodopa or carbidopa, or a combination of levodopa and carbidopa.
